(19) United States
(12) Patent Application Publication
(10) Pub. No.: US 2026/0272497 A1
(43) Pub. Date: Sep. 17, 2026

(54) CORDLESS ENERGY-BASED SURGICAL INSTRUMENTS INCLUDING REUSABLE AND DISPOSABLE PORTIONS

(71) Applicant: Covidien LP, Mansfield, MA (US)

(72) Inventors: Matthew S. COWLEY, Lafayette, CO (US); Michael B. LYONS, Lafayetter, CO (US)

(21) Appl. No.: 19/471,223

(22) PCT Filed: Apr. 4, 2024
(86) PCT No.: PCT/US2024/023081

§ 371 Date: Sep. 30, 2025

Related U.S. Application Data
(60) Provisional application No. 63/457,555, filed on Apr. 6, 2023.
(60) Provisional application No. 63/525,917, filed on Jul. 10, 2023.

(51) Int. Cl. A61B 17/32; A61B 17/00

(52) U.S. Cl. CPC A61B 17/320092; A61B 2017/0046; A61B 2017/00734; A61B 2017/320095

(57) ABSTRACT

A surgical instrument includes a housing including a sealed compartment and an exposed shell, and electronics sealed within the sealed compartment of the housing. The surgical instrument further includes an engagement mechanism configured to releasably engage an elongate assembly with the housing, an actuation lever configured to mechanically actuate an end effector of the elongate assembly, and/or an activation assembly including an activation button movable relative to the housing and one or more electrical switches configured to detect activation of the activation button and transmit the detected activation to the electronics. The housing, the engagement mechanism, the actuation lever, and/or the activation assembly are configured to withstand autoclave sterilization.

### CROSS-REFERENCE TO RELATED APPLICATIONS

This application claims the benefit of, and priority to, U.S. Provisional Patent Application No. 63/457,555, filed on Apr. 6, 2023, and U.S. Provisional Patent Application No. 63/525,917, filed on Jul. 10, 2023, the entire contents of each of which are hereby incorporated herein by reference.

### FIELD

The present disclosure relates to surgical instruments and, more particularly, to cordless energy-based surgical instruments including reusable and disposable portions.

### BACKGROUND

Cordless surgical instruments advantageously provide increased portability and maneuverability and avoid the logistical challenges of positioning, maintaining, and detangling cables that tether surgical instruments to consoles and/or power sources. Cordless energy-based surgical instruments, for example, include on-board electronics (e.g., power and generator functionality), thus obviating the need to tether the instruments to surgical generators.

Surgical instruments can be generally categorized as reusable instruments (i.e., instruments that may be used again after sterilization, cleaning, or other suitable preparation), disposable instruments (i.e., instruments intended to be discarded after use in an environmentally safe fashion), and reposable instruments (i.e., instruments having at least one disposable portion and at least one reusable portion).

### SUMMARY

Surgical instruments that are reusable or include reusable portions are advantageous in that they reduce waste and reduce cost by enabling reuse of the instruments or portions thereof. However, enabling reuse of a surgical instrument or a portion thereof requires sterilization, cleaning, and/or other preparation, in order to inhibit contamination of the sterile surgical field. This sterilization, cleaning, and/or other preparation may be particularly challenging with respect to sensitive components such as, for example, batteries, surgical generators, electrical connectors, circuit boards, etc.

As used herein, the term “distal” refers to the portion that is described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations and tolerances, up to and including plus or minus 10 percent. Further, any or all of the aspects described herein, to the extent consistent, may be used in conjunction with any or all of the other aspects described herein.

Provided in accordance with aspects of the present disclosure is a surgical instrument including a housing and an activation assembly.

In aspects of the present disclosure, the surgical instrument further includes electronics. Alternatively, the electronics may be disposed in a remote generator connected to the surgical instrument via a surgical cable. In aspects, the housing may include a sealed compartment and an exposed shell and/or may be configured to withstand autoclave sterilization. The housing may additionally or alternatively be configured to releasably engage an elongate assembly including an end effector. The electronics, in aspects where provided as part of the surgical instrument, may be sealed within the sealed compartment of the housing such that the sealed compartment protects the electronics to enable autoclave sterilization of the housing without damaging the electronics.

The activation assembly is operably coupled to the housing (in aspects, to the exposed shell of the housing). The activation assembly includes an activation button movable relative to the housing between an un-activated position and at least one activated position, a tactile assembly configured to provide a perceptible output indicating a position of the activation button, and at least one electrical switch configured to detect activation of the activation button. The at least one electrical switch is electrically coupled to the electronics (whether disposed on or within the housing or in a remote generator connected to the surgical instrument via a surgical cable) and configured to transmit the detected activation to the electronics. With the elongate assembly engaged with the housing, the electronics, in response to receipt of the detected activation, are configured to energize the end effector of the elongate assembly to treat tissue with energy. The activation assembly may be configured to withstand autoclave sterilization.

In an aspect of the present disclosure, the tactile assembly includes at least one clicker tab coupled to the housing and extending into an actuation path of the activation button such that the at least one clicker tab provides the perceptible output in response to movement of the activation button from the un-activated position to one of the at least one activated positions.

In another aspect of the present disclosure, the tactile assembly includes at least one bistable spring disposed in an actuation path of the activation button such that the at least one bistable spring provides the perceptible output in response to movement of the activation button from the un-activated position to one of the at least one activated positions.

In still another aspect of the present disclosure, the tactile assembly includes at least one mechanical dome disposed in an actuation path of the activation button such that the at least one mechanical dome provides the perceptible output in response to movement of the activation button from the un-activated position to one of the at least one activated positions.

In yet another aspect of the present disclosure, the tactile assembly includes a clicker tab coupled to the activation button and configured to interact with a portion of the housing in response to movement of the activation button from the un-activated position to one of the at least one activated positions such that the at least one clicker tab provides the perceptible output in response to the movement of the activation button from the un-activated position to the one of the at least one activated positions. In such aspects, the clicker tab may be integrally formed with the activation button. Alternatively or additionally, the activation button may include a biasing spring integrally formed with the activation button.

Another surgical instrument provided in accordance with the present disclosure includes a housing, an engagement mechanism, and an actuation lever.

In aspects of the present disclosure, the surgical instrument further includes electronics. Alternatively, the electronics may be disposed in a remote generator connected to the surgical instrument via a surgical cable. The housing, in aspects, may include a sealed compartment and an exposed shell and/or may be configured to withstand autoclave sterilization. The electronics, where provided as part of the surgical instrument, may be sealed within the sealed compartment of the housing such that the sealed compartment protects the electronics to enable autoclave sterilization of the housing without damaging the electronics.

The engagement mechanism is operably coupled to the exposed shell of the housing and configured to releasably engage an elongate assembly with the housing. The engagement mechanism may be configured to withstand autoclave sterilization. The actuation lever is operably coupled to the exposed shell of the housing, configured to couple to the elongate assembly when the elongate assembly is engaged with the housing, and configured to mechanically actuate an end effector of the elongate assembly when the elongate assembly is engaged with the housing. The actuation lever may be configured to withstand autoclave sterilization. When the elongate assembly is engaged with the housing, the electronics (whether part of the surgical instrument or remote) are configured to energize the end effector of the elongate assembly to treat tissue with energy.

In an aspect of the present disclosure, the electronics include an ultrasonic generator and an ultrasonic transducer. In such aspects, an ultrasonic horn may be engaged with the ultrasonic transducer within the sealed compartment and extend into the exposed shell. The ultrasonic horn may further define a distal connector disposed within the exposed shell and configured to releasably engage an ultrasonic waveguide of the elongate assembly.

In another aspect of the present disclosure, the electronics include an ultrasonic generator, and the surgical instrument further includes an ultrasonic transducer assembly releasably rotatably engagable with the housing.

In still another aspect of the present disclosure, first and second electrical contacts are disposed within the exposed shell of the housing and electrically coupled to the ultrasonic generator. The ultrasonic transducer assembly is rotatably engaged with the housing. The first and second electrical contacts are configured to maintain electrical contact with first and second ring contacts, respectively, of the ultrasonic transducer assembly regardless of a rotational orientation of the ultrasonic transducer assembly relative to the housing.

In yet another aspect of the present disclosure, the first and second ring contacts are disposed annularly about a circumference of the ultrasonic transducer assembly. Alternatively, the first and second ring contacts are concentrically disposed about an ultrasonic horn of the ultrasonic transducer assembly on a distal face of the ultrasonic transducer assembly.

In still yet another aspect of the present disclosure, at least one contact is disposed within the exposed shell and electrically coupled to the electronics. The at least one contact is configured to detect engagement of the elongate assembly with the housing. In such aspects, the electronics may be configured to at least one of read information from or write information to the elongate assembly via the at least one contact.

In another aspect of the present disclosure, the engagement mechanism includes at least one lock arm movable relative to the housing between a disengaged position, permitting insertion and removal of the elongate assembly to and from the housing, and an engaged position, engaging the elongate assembly with the housing. In such aspects, the engagement mechanism may further include at least one biasing spring biasing the at least one lock arm towards the engaged position.

In another aspect of the present disclosure, the engagement mechanism includes a slider slidable about a circumference of the housing between a disengaged position, permitting insertion and removal of the elongate assembly to and from the housing, and an engaged position, engaging the elongate assembly with the housing.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a perspective view of a cordless, ultrasonic surgical instrument provided in accordance with the present disclosure;

FIG. 2 is a perspective view of the surgical instrument of FIG. 1 with a disposable portion of the surgical instrument shown disengaged from a reusable portion of the surgical instrument;

FIG. 3 is a side, longitudinal, cross-sectional view of the reusable portion of the surgical instrument of FIG. 1 including a nozzle of the disposable portion engaged therewith;

FIG. 4 is a perspective view of a cordless, Radio Frequency (RF) surgical instrument provided in accordance with the present disclosure;

FIG. 5 is a side, longitudinal, cross-sectional view of the reusable portion of the surgical instrument of FIG. 4 including a nozzle of the disposable portion engaged therewith;

FIG. 6 is an enlarged, top, longitudinal, cross-sectional view illustrating releasable engagement of a nozzle of a disposable portion of a surgical instrument with a reusable portion of the surgical instrument in accordance with the present disclosure;

FIG. 7 is a side, longitudinal, cross-sectional view of an activation assembly of a reusable portion of a surgical instrument in accordance with the present disclosure;

FIG. 8A is a schematic illustration of a tactile assembly configured for use with the activation assembly of FIG. 7;

FIG. 8B is a schematic illustration of another tactile assembly configured for use with the activation assembly of FIG. 7;

FIG. 9 is a side, longitudinal, cross-sectional view of another activation assembly configured for use with a surgical instrument in accordance with the present disclosure;

FIG. 10 is a side, longitudinal, cross-sectional view of still another activation assembly configured for use with a surgical instrument in accordance with the present disclosure;

FIG. 11 is a side view of a trigger of an activation button configured for use with a surgical instrument in accordance with the present disclosure;

FIG. 12 is a side, longitudinal, partial cross-sectional view of a portion of another cordless, ultrasonic surgical instrument provided in accordance with the present disclosure;

FIG. 13 is an enlarged view of the area of detail indicated as “13” in FIG. 12;

FIG. 14 is a side, longitudinal, partial cross-sectional view of a portion of yet another cordless, ultrasonic surgical instrument provided in accordance with the present disclosure;

FIGS. 15 and 16 are transverse, cross-sectional and perspective, partial cut-away views, respectively, of an engagement mechanism configured to releasably engage a disposable portion of a surgical instrument with a reusable portion of the surgical instrument in accordance with the present disclosure; and

FIGS. 17 and 18 are transverse, cross-sectional and perspective, transverse, cross-sectional views, respectively, of another engagement mechanism configured to releasably engage a disposable portion of a surgical instrument with a reusable portion of the surgical instrument in accordance with the present disclosure.

### DETAILED DESCRIPTION

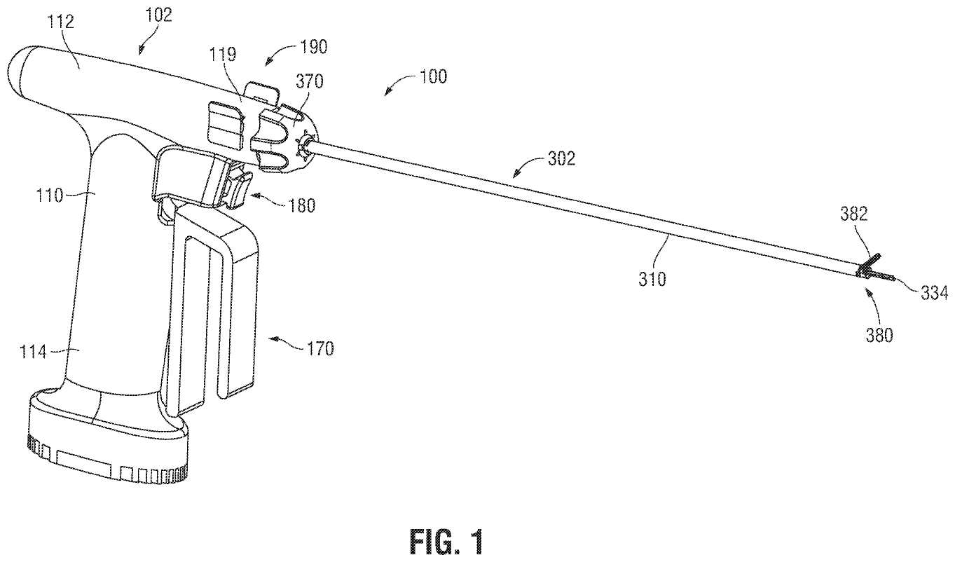

Referring to FIGS. 1-3, a cordless ultrasonic surgical instrument provided in accordance with the aspects and features of the present disclosure is shown generally identified by reference numeral 100. Surgical instrument 100 generally includes a handle assembly 102 and an elongate assembly 302 that is configured to releasably engage handle assembly 102. Handle assembly 102, in aspects, is configured as a reusable portion such that, after a use of handle assembly 102 in a surgical procedure, handle assembly 102 is capable of being sterilized (e.g., in an autoclave), cleaned, and/or otherwise prepared for reuse. Elongate assembly 302, in aspects, is configured as a disposable portion configured to be used in a single surgical procedure and safely discarded or returned for reprocessing after such use.

Handle assembly 102 includes a housing 110 defining a barrel portion 112 and a fixed handle portion 114 depending from barrel portion 112 thus defining a pistol grip configuration, although other configurations are also contemplated. Housing 110 includes a sealed compartment 116 and an exposed shell 118. Sealed compartment 116 supports and encloses an ultrasonic transducer 120, a power source 130, a surgical generator 140, a charging interface 150, and an electrical connection assembly 160. Sealed compartment 116 may be hermetically sealed, aseptically sealed, otherwise sealed, and/or otherwise configured to enable handle assembly 102 to withstand autoclaving and/or other sterilization without damaging the internal components within sealed compartment 116 (some or all of which are incapable of withstanding autoclaving and/or other sterilization without the isolation and protection provided by sealed compartment 116). Housing 110 is formed from suitable materials and suitably constructed to withstand autoclaving and/or other sterilization, cleaning, or preparation.

Exposed shell 118 of handle assembly 102 operably supports an actuation lever 170, an activation assembly 180, and an engagement mechanism 190 and is configured to releasably receive a portion of elongate assembly 302 to releasably engage elongate assembly 302 with handle assembly 102. The interior of exposed shell 118 is exposed to the environment, at least in the absence of elongate assembly 302 engaged therein. Exposed shell 118 defines a distal nose 119 through which a portion of elongate assembly 302 is inserted to releasably engage elongate assembly 302 with exposed shell 118, as detailed below.

An ultrasonic horn 210 is engaged with ultrasonic transducer 120 within sealed compartment 116 and extends from sealed compartment 116 into exposed shell 118 to enable operable engagement of elongate assembly 302 with ultrasonic horn 210 and, thus, ultrasonic transducer 120. Ultrasonic horn 210 defines a distal connector 212, e.g., a threaded female receiver, to enable engagement of ultrasonic horn 210 with waveguide 330 of elongate assembly 302, as detailed below. Ultrasonic horn 210 is constructed from suitable materials to withstand autoclaving and/or other sterilization, cleaning, or preparation.

Continuing with reference to FIGS. 1-3, handle assembly 102 may include one or more internal dividers 220 configured to separate sealed compartment 116 from exposed shell 118. The one or more internal dividers 220 may include one or more mechanical pass-throughs such as, for example, a tunnel 222 configured to receive ultrasonic horn 210 therethrough such that, as noted above, ultrasonic horn 210 extends from sealed compartment 116 into exposed shell 118. Tunnel 222 may define an annular recess 224 configured to receive an annular flange 214 of ultrasonic horn 210 to rotatably mount ultrasonic horn 210 within tunnel 222 and relative to housing 110 of handle assembly 102. One or more seals 226 disposed about an exterior surface of ultrasonic horn 210, disposed on an interior surface of tunnel 222, and/or disposed between tunnel 222 and ultrasonic horn 210 is provided to maintain the sealed condition of sealed compartment 116 despite ultrasonic horn 210 passing through tunnel 222 from sealed compartment 116 into exposed shell 118. The one or more internal dividers 220 may be formed integrally with housing 110 and/or similarly formed from suitable materials and of suitable construction to withstand autoclaving and/or other sterilization, cleaning, or preparation.

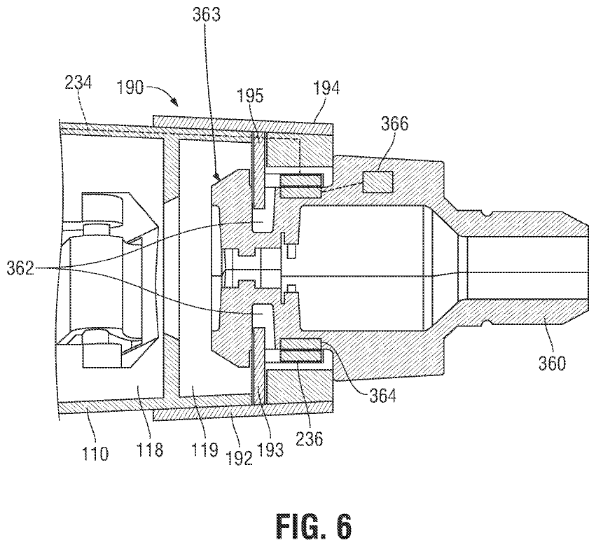

One or more electrical connectors 232, e.g., electrical wires, electrical contacts, electrically conductive elements, combinations thereof, and/or or other suitable electrical pathways, may be embedded within the material forming housing 110 and/or disposed within internal sealed conduits within housing 110 to provide electrical connection to and from sealed compartment 116 such as, for example, to enable electrical connection between activation assembly 180 and electrical connection assembly 160 (or directly between activation assembly 180 and power source 130 and/or surgical generator 140). Additionally or alternatively, one or more of such electrical connectors 234 (FIG. 6) may enable electrical connection between surgical generator 140 and elongate assembly 302 via one or more electrical contacts 236 (disposed on or within exposed shell 118 of housing 110). Such electrical connections may enable communication of identification (ID) information, sensor feedback (e.g., from sensors associated with end effector 380 (FIG. 1)), activation signals, etc.

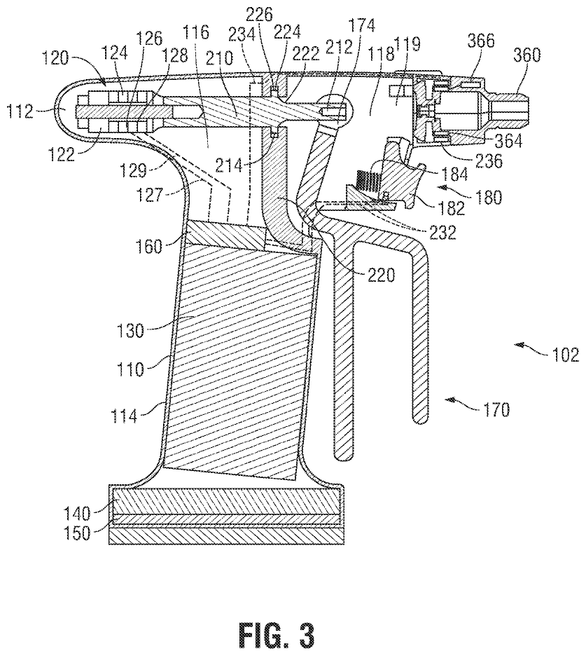

With reference to FIG. 3, ultrasonic transducer 120 includes an end mass 122, a piezoelectric stack 124 held in compression between end mass 122 and ultrasonic horn 210 (directly or indirectly), and first and second electrodes 126, 128 disposed between the piezoelectric elements of the piezoelectric stack 124. First and second electrodes 126, 128 are rotatably coupled to connectors 127, 129, respectively, to enable drive signals to be communicated to piezoelectric stack 124 to drive ultrasonic transducer 120 regardless of the rotational orientation of ultrasonic transducer 120 relative to housing 110 and connectors 127, 129. More specifically, when driven, piezoelectric stack 124 of ultrasonic transducer 120 converts an AC signal received from surgical generator 140 (via connectors 127, 129 and electrodes 126, 128) into mechanical motion that is output along ultrasonic horn 210. Ultrasonic transducer 120 and the connections thereto, e.g., connectors 127, 129, are disposed within sealed compartment 116 to protect ultrasonic transducer 120 from damage during autoclaving, other sterilization, cleaning, and/or preparation for reuse. In aspects, a rotation knob (not shown) engaged with ultrasonic transducer 120 extends from sealed compartment 116 (as a pass-through mechanical component similarly as detailed with respect to ultrasonic horn 210) to enable rotation of ultrasonic transducer 120 relative to housing 110 from the exterior of housing 110. This rotation knob may also be used to hold ultrasonic transducer 120 in position to enable threaded engagement of waveguide 330 therewith, e.g., upon rotation of rotation knob 370 of elongate assembly 302 relative to ultrasonic transducer 120.

Power source 130 may include a battery having, for example, one or more battery cells, one or more solid state batteries, or any other suitable battery configuration. Power source 130 may alternatively or additionally include an array of array of capacitors or other suitable energy-storage device(s). As noted above, power source 130 is disposed within sealed compartment 116 to protect power source 130 from damage during autoclaving, other sterilization, cleaning, and/or preparation for reuse. Insulation (not shown) may also be provided to protect power source 130 (and/or other electronics) from heat associated with autoclaving and/or other processes in preparation for reuse.

Surgical generator 140, powered by power source 130, is configured to control the high voltage AC signal supplied to ultrasonic transducer 120. For example, surgical generator 140 may be configured to control the drive signal output to ultrasonic transducer 120 in accordance with a “LOW” power mode or a “HIGH” power mode, e.g., depending upon feedback received from activation assembly 180 as to the activated position of activation button 182. As noted above, surgical generator 140 is disposed within sealed compartment 116 to protect surgical generator 140 from damage during autoclaving, other sterilization, cleaning, and/or preparation for reuse.

Charging interface 150 is configured to enabling charging of power source 130 from the exterior of handle assembly 102 and without compromising sealed compartment 116. Charging interface 150 may include, for example, an inductive coupling configured to interface with an inductive coupling of a battery charger (not shown) to transfer power and/or data therebetween, thus enabling the charging of power source 130 and/or the communication of data between power source 130, surgical generator 140, and/or other components of handle assembly 102 with the battery charger. As noted above, charging interface 150 is disposed within sealed compartment 116 to protect charging interface 150 from damage during autoclaving, other sterilization, cleaning, and/or preparation for reuse.

Electrical connection assembly 160 may include, for example, one or more flex circuits, or other suitable electrical connections, to establish electrical communication between ultrasonic transducer 120, power source 130, surgical generator 140, charging interface 150, activation assembly 180, and/or ID contact(s) 236. As noted above, electrical connection assembly 160 is disposed within sealed compartment 116 to protect electrical connection assembly 160 from damage during autoclaving, other sterilization, cleaning, and/or preparation for reuse.

Continuing with reference to FIG. 3, actuation lever 170 extends from housing 110 of handle assembly 102 adjacent fixed handle portion 114 of housing 110. Actuation lever 170, more specifically, is pivotably coupled to housing 110 within exposed shell 118 and extends from exposed shell 118 exteriorly of housing 110 to enable manual manipulation of actuation lever 170 by a user. Actuation lever 170 includes a bifurcated drive portion 174 disposed within exposed shell 118 of housing 110. Bifurcated drive portion 174 is configured to engage outer drive shaft 310 of elongate assembly 302 upon engagement of elongate assembly 302 with handle assembly 102 such that pivoting of actuation lever 170 relative to housing 110 between an initial position, wherein actuation lever 170 is spaced apart from fixed handle portion 114 of housing 110, and an actuated position, wherein actuation lever 170 is more closely approximated relative to fixed handle portion 114 of housing 110, translates outer drive shaft 310 of elongate assembly 302 to actuate elongate assembly 302, as detailed below. Actuation lever 170 is formed from suitable materials and suitably constructed so as to withstand autoclaving, other sterilization, cleaning, and/or other preparation for reuse.

An activation assembly 180 includes an activation button 182 pivotably supported by housing 110 and extending into exposed shell 118 of housing 110, and a biasing member 184, e.g., a compression spring, disposed within exposed shell 118 of housing 110 and positioned to bias activation button 182 away from exposed shell 118 of housing 110. Activation button 182 is movable, e.g., pivotable, against the bias of biasing member 184, from an un-activated position, wherein activation button 182 protrudes farther from exposed shell 118 of housing 110, to one or more activated positions, wherein activation button 182 extends farther into exposed shell 118 of housing 110. In aspects, activation button 182 includes first and second activated positions disposed in-line with one another such that activation button 182 is movable towards exposed shell 118 of housing 110 in the same direction from the un-activated position to the first activated position and from the first activated position to the second activated position. However, other configurations are also contemplated. In aspects, the first activated position corresponds to the “LOW” power mode of ultrasonic transducer 120, while the second activated position corresponds to the “HIGH” power mode of ultrasonic transducer 120. Other modes are also contemplated. Activation button 182 and biasing member 184 are constructed so as to withstand autoclaving, other sterilization, cleaning, and/or other preparation for reuse. The electronic components of activation assembly 180 that enable communication of the position of activation button 182 to power source 130 and/or surgical generator 140, as detailed below, are formed from suitable materials and suitably constructed so as to withstand autoclaving, other sterilization, cleaning, and/or other preparation for reuse and/or are protected by housing 110, thus inhibiting damage during autoclaving, other sterilization, cleaning, and/or other preparation for reuse. However, the activation assemblies provided in accordance with the present disclosure are contemplated for use in any suitable surgical instruments including fully or partially reusable or disposable surgical instruments, including corded instruments.

Referring again to FIGS. 1-3, and with momentary reference to FIG. 6, engagement mechanism 190 is disposed at distal nose 119 of housing 110 and is configured to enable releasable engagement of elongate assembly 302 with distal nose 119 of housing 110. An exemplary configuration of engagement mechanism 190 is detailed below, although other configurations are also contemplated. As shown in FIG. 6, ID contact 236 may be in the form of an outer annular ring disposed within distal nose 119 and configured to align with a corresponding ID contact 364 in the form of an inner annular ring disposed on elongate assembly 302 upon engagement of elongate assembly 302 with handle assembly 102 to enable communication therebetween. In aspects, plural pairs of cooperating contacts 236, 364 may be provided. ID contacts 236, 364 enable detection of the presence of an elongate assembly 302 and/or communication of data between elongate assembly 302 and handle assembly 102. Communicating data may include, for example, accessing one or more memories 366 of elongate assembly 302, e.g., an EEPROM, reading a circuit property (e.g., resistance) corresponding to information, or otherwise reading/writing data to/from elongate assembly 302. With respect to memory 366 of elongate assembly 302, which may be disposed on or within nozzle 360, memory 366 may be a read-only memory storing identifying information associated with elongate assembly 302 that can be read by handle assembly 102, e.g., unique ID, device type, lot number, manufacture date, configuration data, features, settings, authentication information, etc. Memory 366 of elongate assembly 302 may alternatively be configured as a read/write memory to enable information to be read/written to/from memory 366. Such information may include, for example, the information noted above, a use count, usage information, a use flag indicating that elongate assembly 302 has been used, an error log, etc. Engagement mechanism 190 is formed from suitable materials and of suitable construction to withstand autoclaving and/or other sterilization, cleaning, or preparation. However, the engagement assemblies provided in accordance with the present disclosure are contemplated for use in any suitable surgical instruments including fully or partially reusable or disposable surgical instruments, including corded instruments.

As noted above, as an alternative or in addition to communicating with an EEPROM, other communication between elongate assembly 302 and handle assembly 102 may be established via contacts 236, 364 such as, for example, to communicate sensor feedback data, activation signals, etc. Further, with respect to any communication between elongate assembly 302 and handle assembly 102, contacts 236, 364 may be replaced with wireless communication components such as, for example, to enable RFID communication, inductive or magnetic communication, optical communication, etc.

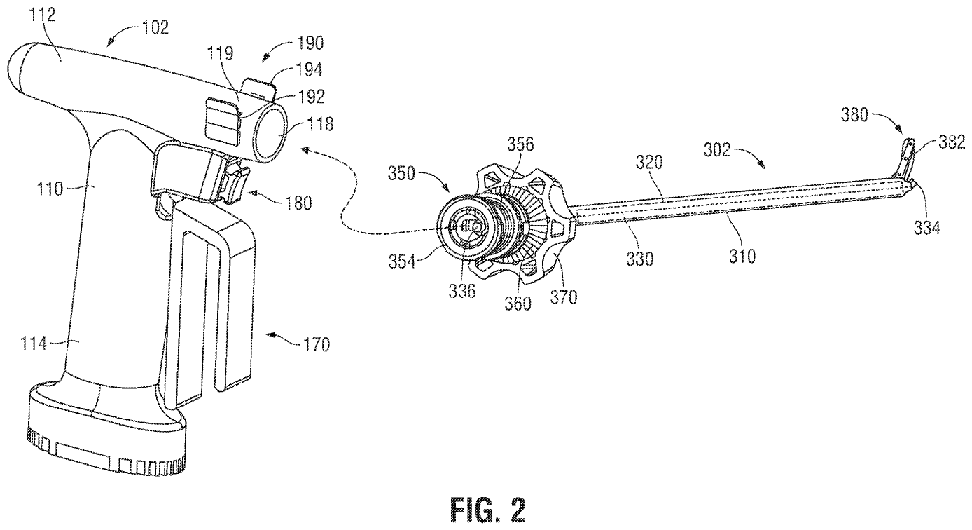

With reference to FIGS. 1 and 2, elongate assembly 302 includes an outer drive shaft 310, an inner support shaft 320 disposed within outer drive shaft 310 and about which outer drive shaft 310 is configured to translate, a waveguide 330 extending through inner support shaft 320, a drive assembly 350 disposed about outer drive shaft 310 and configured to operably couple outer drive shaft 310 with bifurcated drive portion 174 of actuation lever 170, a nozzle 360 disposed about outer drive shaft 310 and operably coupled to waveguide 330, a rotation knob 370 operably disposed about nozzle 360, and an end effector 380 disposed at the distal end of inner support shaft 320. Elongate assembly 302, as detailed below, is configured to releasably engage handle assembly 102 such that mechanical ultrasonic motion output from ultrasonic transducer 120 is transmitted along ultrasonic horn 210 and waveguide 330 to end effector 380 for treating tissue therewith, such that actuation lever 170 is selectively actuatable to manipulate end effector 380, and such that rotation knob 370 is selectively rotatable to rotate elongate assembly 302 relative to handle assembly 102.

Outer drive shaft 310 is slidably disposed about inner support shaft 320. Outer drive shaft 310 and inner support shaft 320 are operably coupled to jaw member 382 of end effector 380 at the distal end thereof. For example, jaw member 382 may include one or more jaw flanges pivotably mounted at the distal end of inner support shaft 320 to pivotably couple jaw member 382 to inner support shaft 320, and a jaw foot extending through an aperture defined within outer drive shaft 310 at the distal end thereof such that proximal translation of outer drive shaft 310 about inner support shaft 320 and relative to end effector 380 pivots jaw member 382 from an open position to a clamping position. Other suitable actuation configurations are also contemplated. Further, the above-detailed configuration may be reversed, e.g., wherein the outer shaft is the support shaft and wherein the inner shaft is the drive shaft.

Waveguide 330 extends through inner support shaft 320. Waveguide 330 defines a blade 334 at a distal end thereof and a proximal connector 336 at a proximal end thereof. Waveguide 330 and inner and outer shafts 310, 320 are rotationally fixed relative to nozzle 360 and rotation knob 370 is fixedly engaged about nozzle 360 such that rotation of rotation knob 370 rotates nozzle 360, waveguide 330, and inner and outer shafts 310, 320 relative to housing 110 of handle assembly 102. Inner support shaft 320 is fixedly engaged with nozzle 360 such that, upon rotatable engagement of nozzle 360 with handle assembly 102, as detailed below, inner support shaft 320 is likewise rotatably engaged with handle assembly 102.

Blade 334 extends distally from inner support shaft 320 and forms part of end effector 380 in that blade 334 is positioned to oppose jaw member 382 such that pivoting of jaw member 382 from the open position to the clamping position enables clamping of tissue between jaw member 382 and blade 334. Blade 334 and jaw member 382 define curved configurations wherein the directions of movement of jaw member 382 between the open and clamping positions are perpendicular to the directions of curvature. However, it is also contemplated that blade 334 and jaw member 382 define a straight configuration or that blade 334 and jaw member 382 curve in either of the directions of movement of jaw member 382. Jaw member 382 may include a structural jaw frame supporting a compliant jaw liner configured to oppose blade 334 in the clamping position.

Proximal connector 336 of waveguide 330 is configured to enable engagement of waveguide 330 with ultrasonic horn 210 such that mechanical motion produced by ultrasonic transducer 120 is transmitted along waveguide 330 to blade 334 for treating tissue clamped between blade 334 and jaw member 382 or positioned adjacent to blade 334. Proximal connector 336 may include, for example, a threaded male shaft that is configured for threaded engagement within threaded female receiver of distal connector 212 of ultrasonic horn 210. With waveguide 330 engaged with ultrasonic horn 210, rotation of rotation knob 370 also rotates ultrasonic transducer 120 and ultrasonic horn 210 within and relative to housing 110 of handle assembly 102.

Continuing with reference to FIGS. 2-3, drive assembly 350 is disposed about outer drive shaft 310 and configured to operably couple outer drive shaft 310 with bifurcated drive portion 174 of actuation lever 170 upon engagement of elongate assembly 302 with handle assembly 102. More specifically, drive assembly 350 includes a collar 354 configured for receipt and releasable engagement within bifurcated drive portion 174 of actuation lever 170 such that actuation of actuation lever 170 translates collar 354 to thereby translate outer drive shaft 310. Drive assembly 350 may further include a biasing member 356 (e.g., a spring) coupled between collar 354 and outer drive shaft 310 to regulate a force transmitted from actuation lever 170 to outer drive shaft 310, thereby controlling a force applied to tissue clamped between jaw member 382 and blade 334. In other configurations, biasing member 356 is omitted. Collar 354 is rotatably engaged within bifurcated drive portion 174 to enable rotation of drive assembly 350 and, thus, elongate assembly 302 relative to handle assembly 102 when elongate assembly 302 is engaged with handle assembly 102.

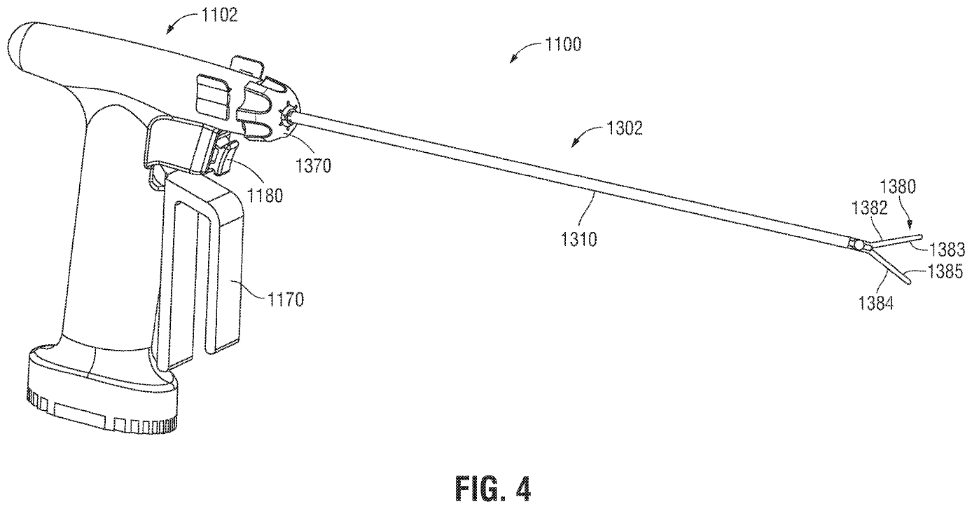

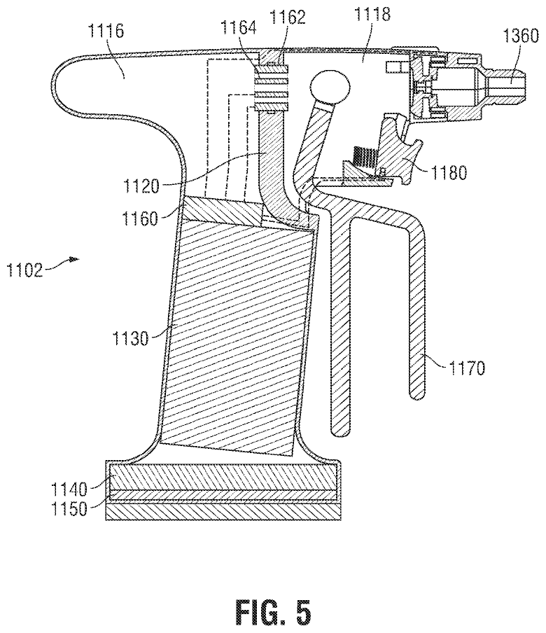

Turning to FIGS. 4 and 5, a cordless Radio Frequency (RF) surgical instrument provided in accordance with the aspects and features of the present disclosure is shown generally identified by reference numeral 1100. Surgical instrument 1100 generally includes a handle assembly 1102 and an elongate assembly 1302 that is configured to releasably engage handle assembly 1102. Handle assembly 1102, in aspects, is configured as a reusable portion such that, after a use of handle assembly 1102 in a surgical procedure, handle assembly 1102 is capable of being sterilized (e.g., in an autoclave), cleaned, and/or otherwise prepared for reuse. Elongate assembly 1302, in aspects, is configured as a disposable portion configured to be used in a single surgical procedure and safely discarded after such use. Surgical instrument 1100 is similar to and may include any of the features of surgical instrument 100 (FIG. 1) except as explicitly contradicted below. Accordingly, only difference between surgical instrument 1100 and surgical instrument 100 (FIG. 1) are described in detail below while similarities are summarily described or omitted entirely.

Sealed compartment 1116 of handle assembly 1102 supports and encloses a power source 1130, a surgical generator 1140, a charging interface 1150, and an electrical connection assembly 1160. Electrical connection assembly 1160 includes first and second electrical connectors 1162, 1164 configured as pass-through electrical connections that extend from sealed compartment 1116 into exposed shell 1118 for connection of first and second electrical connectors 1162, 1164 with corresponding electrical pathways (not shown) of elongate assembly 1302 to couple surgical generator 1140 with jaw members 1382, 1384 of elongate assembly 1302 to enable the conducting of RF energy in a bipolar configuration between jaw members 1382, 1384 and through tissue grasped therebetween to seal, cut, and/or otherwise treat tissue. First and second electrical connectors 1162, 1164 may be configured as inner and outer annular ring connectors extending through divider 1120 as pass-through electrical connectors.

Elongate assembly 1302 includes a shaft assembly 1310 (e.g., including a support shaft and a drive shaft), a nozzle 1360 disposed about the proximal end portion of shaft assembly 1310, a rotation knob 1370 engaged about nozzle 1360, and an end effector assembly 1380 supported at the distal end portion of shaft assembly 1310. End effector assembly 1380 includes first and second jaw members 1382, 1384, each defining an electrically conductive surface 1383, 1385, respectively. At least one of the first or second jaw members 1382, 1384 is movable relative to the other, e.g., upon actuation of the drive shaft of shaft assembly 1310, between a spaced-apart position and an approximated position for grasping tissue between electrically conductive surfaces 1383, 1385. A drive assembly (not shown) operably couples shaft assembly 1310 with actuation lever 1170 upon engagement of elongate assembly 1302 with handle assembly 1102. Activation assembly 1180 initiates the supply of energy to electrically conductive surfaces 1383, 1385 to treat tissue and may include one or more activated positions corresponding to one or more modes such as, for example, tissue cutting, tissue sealing, etc.

In aspects, elongate assembly 1302 further includes a knife assembly (not shown) and handle assembly 1102 further includes a knife lever (not shown). The knife assembly may operably couple to the knife lever upon engagement of elongate assembly 1302 with handle assembly 1102, e.g., similarly as detailed above with respect to the actuation lever and drive assembly, to enable advancement of a knife (not shown) of the knife assembly between jaw members 1382, 1384 to cut tissue grasped therebetween upon activation of the knife lever. Alternatively or additionally, an energy-based cutter (not shown) such as, for example, a cutting electrode or a thermal cutter, may be provided and coupled to surgical generator 1140 for energy-based cutting of tissue grasped between jaw members 1382, 1384.

Surgical instruments in accordance with the present disclosure may also include RF and ultrasonic functionality, combining the features of surgical instruments 100, 1100 (FIGS. 1 and 4, respectively) as detailed above. In such aspects, separate surgical generators may be provided within the sealed compartment or a combined generator may be provided. Further, RF energy may be conducted between the jaw member and ultrasonic blade (e.g., in a bipolar configuration), in a monopolar configuration from the jaw member or ultrasonic blade to a remote return device, or in any other suitable manner.

With reference to FIG. 6, in conjunction with FIG. 1, engagement mechanism 190 of surgical instrument 100 is described, keeping in mind that engagement mechanism 190 may likewise be used with surgical instrument 1100 (FIG. 4) and/or any other suitable surgical instrument. Engagement mechanism 190 includes opposing lock tabs 192, 194 extending from opposite sides of distal nose 119 to enable manual manipulation by a user. Engagement mechanism 190 further includes lock fingers 193, 195 extending from respective lock tabs 192, 194 into distal nose 119. Lock tabs 192, 194 are configured such that inward squeezing of the free ends of lock tabs 192, 194 retracts lock fingers 193, 195 from the internal passage through distal nose 119 and such that release of lock tabs 192, 194 returns lock fingers 193, 195 inwardly to extend into the internal passage through distal nose 119. Further, with lock fingers 193, 195 extending inwardly into the internal passage through distal nose 119, insertion of elongate assembly 302 through distal nose 119 moves an annular cam surface 363 (or surfaces) defined on a proximal end of nozzle 360 (or other suitable component) of elongated assembly 302 into contact with the free ends of lock fingers 193, 195 to urge lock fingers 193, 195 outwardly, thus permitting further insertion of elongated assembly 302 into distal nose 119. Alternatively, lock tabs 192, 194 may be squeezed inwardly from the free ends thereof to retract lock fingers 193, 195 prior to insertion of elongate assembly 302.

Once elongate assembly 302 is inserted through distal nose 119 and into the internal passageway defined therein (whereby annular cam surface 363 of nozzle 360 clears lock fingers 193, 195, lock tabs 192, 194 are returned under bias (or released) such that lock fingers 193, 195 extend inwardly into distal nose 119 and into engagement within an annular channel 362 defined within nozzle 360, thereby rotatably engaging elongate assembly 302 with handle assembly 102. A rotation lock feature (not shown), e.g., a torque wrench assembly, may also be provided to lock rotation of ultrasonic transducer 120 to facilitate rotation of elongate assembly 302 relative to ultrasonic transducer 120 for threaded engagement (and threaded disengagement) of ultrasonic horn 210 with waveguide 330 (see FIGS. 1-3). Other suitable releasable engagement mechanisms for rotatably engaging elongate assembly 302 with handle assembly 102 are also contemplated.

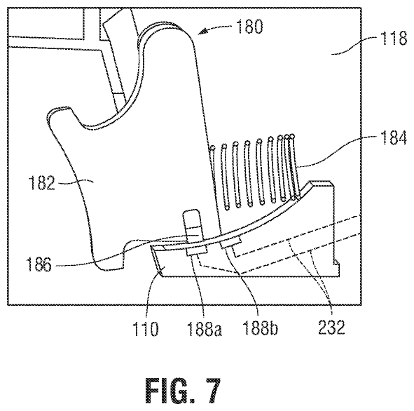

Turning to FIG. 7, as noted above, activation assembly 180 of surgical instrument 100 is described, keeping in mind that activation assembly 180 may likewise be used with surgical instrument 1100 (FIG. 4) and/or any other suitable surgical instrument. Activation assembly 180, as detailed above, includes activation button 182 pivotably supported by housing 110 and extending into exposed shell 118 of housing 110, and biasing member 184 disposed within exposed shell 118 and positioned to bias activation button 182 away from exposed shell 118 of housing 110. As also noted above, these mechanical components of activation assembly 180 are formed of suitable materials and suitably constructed so as to withstand autoclaving, other sterilization, cleaning, and/or other preparation for reuse.

With respect to the electronic components of activation assembly 180 that enable communication of the position of activation button 182 to power source 130 and/or surgical generator 140 (FIG. 3), activation button 182 may be formed from a non-conductive material such as a plastic and includes a magnetic element 186, e.g., a metallic (steel, for example) pin, disposed thereon or therein. Exposed shell 118 of housing 110 includes one or more electrical switches such as first and second sensors 188a, 188b spaced-apart from one another along the actuation path of activation button 182. First and second sensors 188a, 188b may be hall sensors or other suitable non-contact switches such as, for example, capacitive sensors, optical sensors, reed switches, inductive sensors, linear variable differential transformers (LVDTs), ultrasound sensors, etc. First and second sensors 188a, 188b are electrically coupled to power source 130 and/or surgical generator 140 via electrical connectors 232 embedded within the material forming housing 110 and/or disposed within internal sealed conduits within housing 110 to provide electrical connection to and from sealed compartment 116, thus enabling communication of the position of activation button 182 to power source 130 and/or surgical generator 140.

In use, when activation button 182 is moved from the un-activated position to the first activated position, first sensor 188a senses the presence of magnetic element 186, thus enabling power source 130 and/or surgical generator 140 to determine that activation button 182 is disposed in the first activated position and to activate the corresponding mode of operation. Likewise, when activation button 182 is moved to the second activated position, second sensor 188b senses the presence of magnetic element 186, thus enabling power source 130 and/or surgical generator 140 to determine that activation button 182 is disposed in the second activated position and to activate the corresponding mode of operation.

Thus, as detailed above, activation assembly 180 enables mechanical movement of activation button 182 and corresponding sensing to enable power source 130 and/or surgical generator 140 to determine the position of activation button 182, and does so in a manner that enables autoclaving, other sterilization, cleaning, and/or other preparation for reuse of handle assembly 100 without damage to activation assembly 180.

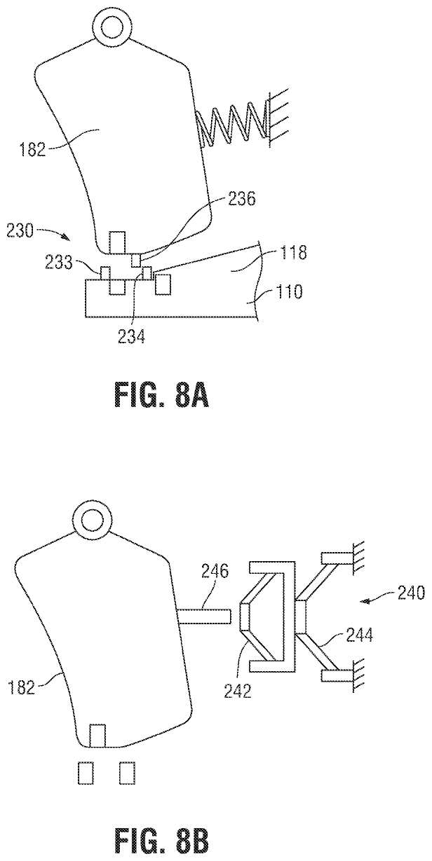

Referring to FIGS. 8A and 8B, activation assembly 180 may further include a tactile assembly 230, 240 (FIGS. 8A and 8B, respectively) configured to provide tactile feedback as to the position of activation button 182. Tactile assemblies 230, 240 (FIGS. 8A and 8B, respectively) are exposed mechanical assemblies formed of materials and constructed to withstand autoclaving, other sterilization, cleaning, and/or other preparation for reuse.

As shown in FIG. 8A, tactile assembly 230 includes first and second clicker tabs 233, 234 extending inwardly from exposed shell 118 of housing 110 into the interior thereof. Further, activation button 182 includes a protrusion 236 extending therefrom. First and second clicker tabs 233, 234 are positioned relative to protrusion 236 such that: upon movement of activation button 182 from the un-activated position to the first activated position, protrusion 236 contacts, deflects, and ultimately traverses first clicker tab 233, thereby producing a first audible and/or tactile click indicating to the user that the first activated position has been achieved; and upon movement of activation button 182 from the first activated position to the second activated position, protrusion 236 contacts, deflects, and ultimately traverses second clicker tab 234, thereby producing a second audible and/or tactile click indicating to the user that the second activated position has been achieved. The first and second audible and/or tactile clicks, in aspects, may be different from one another to enable distinction between the first and second activated positions.

As shown in FIG. 8B, tactile assembly 240 includes first and second bistable springs 242, 244 stacked relative to one another and configured such that a spring force of the first bistable spring 242 is less than a spring force of the second bistable spring 244. Although first bistable spring 242 is shown stacked on second bistable spring 244, the opposite arrangement or other configurations are also contemplated. Activation button 182 includes a protrusion 246 extending therefrom. First and second bistable springs 242, 244 are positioned relative to protrusion 246 such that: upon movement of activation button 182 from the un-activated position to the first activated position, protrusion 246 contacts and deflects first bistable spring 242 from a first position to a second position (without changing the position of second bistable spring 244), thereby producing a first audible and/or tactile output indicating to the user that the first activated position has been achieved; and upon movement of activation button 182 from the first activated position to the second activated position, protrusion 246 urges first bitable spring 242 to contact and deflect second bistable spring 244 from a first position to a second position, thereby producing a second audible and/or tactile output indicating to the user that the second activated position has been achieved. The first and second audible and/or tactile outputs, in aspects, may be different from one another to enable distinction between the first and second activated positions. Springs 242, 244 may alternatively, instead of defining bi-stable configurations, be non-linear and reduce the spring force when the first and second positions are achieved. Such springs 242, 244 may thus serve as the biasing member that biases activation button 182 towards the un-activated position, thus obviating the need for a separate biasing spring; in other aspects, a biasing spring is still provided.

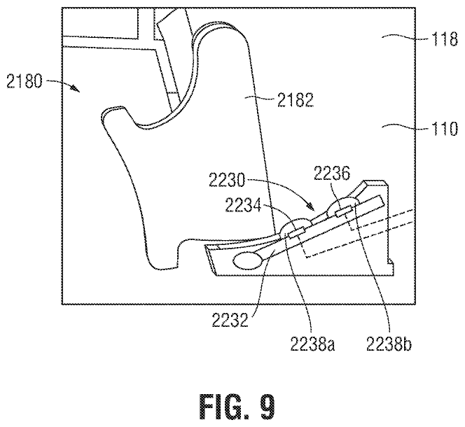

With reference to FIG. 9, another activation assembly 2180 configured for use with surgical instrument 100 (FIG. 1; as detailed hereinbelow), surgical instrument 1100 (FIG. 4), and/or any other suitable surgical instrument is described. Activation assembly 2180 includes an activation button 2182 pivotably supported by housing 110 and extending into exposed shell 118 of housing 110, similarly as detailed above with respect to activation assembly 180 (FIGS. 3 and 7).

Activation assembly 2180 further includes a dome switch assembly 2230 including a sealed circuit board 2232 having first and second electrical switches 2234, 2236 sealed therein. Electrical switches 2234, 2236 are coupled to electrical connectors 232 embedded within the material forming housing 110 and/or disposed within internal sealed conduits within housing 110 to provide electrical connection to and from sealed compartment 116, thus enabling communication of the position of activation button 2182 to power source 130 and/or surgical generator 140 (see FIG. 3). Dome switch assembly 2230 further includes first and second mechanical domes 2238a, 2238b disposed on circuit board 2232 and extending into exposed shell 118 of housing 110. Mechanical domes 2238a, 2238b are formed of materials and constructed so as to withstand autoclaving, other sterilization, cleaning, and/or other preparation for reuse.

Mechanical dome 2238a is positioned such that, when activation button 2182 is moved from the un-activated position to the first activated position, a portion of activation button 2182 contacts and inverts first mechanical dome 2238a to thereby close first electrical switch 2234 and produce a tactile and/or audible output, thus enabling power source 130 and/or surgical generator 140 to determine that activation button 2182 is disposed in the first activated position and indicating to the user the position of activation button 2182. Likewise, second mechanical dome 2238b is positioned such that, when activation button 2182 is moved from the first activated position to the second activated position, a portion of activation button 2182 contacts and inverts second mechanical dome 2238b to thereby close second electrical switch 2236 and produce a tactile and/or audible output, thus enabling power source 130 and/or surgical generator 140 to determine that activation button 2182 is disposed in the second activated position and indicating to the user the position of activation button 2182. The first and second audible and/or tactile outputs, in aspects, may be different from one another to enable distinction between the first and second activated positions.

Further, circuit board 2232 may be coupled to exposed shell 118 of housing 110 in a cantilever configuration wherein circuit board 2232 is biased to intersect the travel path of activation button 2182. In this configuration, as activation button 2182 is moved from the un-activated position to the activated position(s), activation button 2182 contacts and deflects circuit board 2232 against the bias thereof. Thus, upon release of activation button 2182, the bias of circuit board 2232 urges circuit board 2232 back towards its at-rest position to, in turn, urge activation button 2182 back towards the un-activated position. In this manner, circuit board 2232 biases activation button 2182 towards the un-activated position. As an alternative to circuit board 2232, dome switch assembly 2230 may be disposed on any other suitable cantilever support structure to provide the above-detailed bias; or any other suitable structure including dome switch assembly 2230 being disposed on or within housing 110.

In aspects, mechanical domes 2238a, 2238b may be provided for use with any of the other activation assemblies detailed herein, e.g., to provide tactile feedback. In such aspects, mechanical domes 2238a, 2238b may be supported on or within housing 110 or may be disposed on a board 2232 fixed relative to housing 110 or defining a cantilever configuration as detailed above. Although the above switch assemblies are detailed as multi-stage switches, it is also contemplated that the above switch assemblies include one or more single-stage switches in place of or in addition to the above-detailed multi-stage switches.

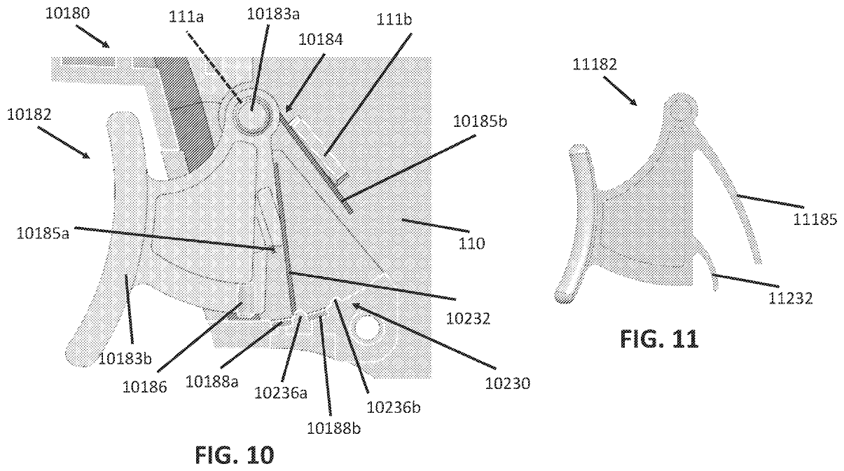

Referring to FIG. 10, another activation assembly 10180 configured for use with surgical instrument 100 (FIG. 1; as detailed hereinbelow), surgical instrument 1100 (FIG. 4), and/or any other suitable surgical instrument is described. Activation assembly 10180 is similar to and may include any of the features of any of the activation assemblies detailed hereinabove, except as explicitly contradicted below. Accordingly, features of activation assembly 10180 similar to the features of any of the activation assemblies detailed hereinabove are summarily described hereinbelow or omitted entirely for purposes of brevity.

Activation assembly 10180 includes an activation button 10182, a biasing spring 10184, a sensor element 10186, first and second sensors 10188a, 10188b, and a tactile assembly 10230 including a clicker tab 10232 and first and second protrusions 10236a, 10236b. Activation button 10182 is pivotably supported by housing 110 and extends from housing 110 to the exterior thereof. More specifically, activation button 10182 may include a pair of opposed pivot bosses 10183a (only one of which is shown in FIG. 10) pivotably received within corresponding pivot recesses 111a (only one of which is shown in FIG. 10) defined within housing 110 and a manipulation portion 10183b extending from housing 110 to an exterior thereof to enable manipulation of activation button 10182 by a user, e.g., between an un-actuated position, first actuated position, and second actuated position.

Biasing spring 10184 is configured as a torsion spring, although other springs are also contemplated. Biasing spring 10184 includes a body (not shown) disposed about one of the pivot bosses 10183a of activation button 10182, a first leg 10185a abutting (and, in aspects, secured to) a portion of activation button 10182, and a second leg 10185b abutting a stop surface 111b disposed within housing 110. First and second legs 10185a, 10185b are biased apart from one another such that manipulation portion 10183b is biased outwardly from housing 110 towards the un-actuated position (as shown in FIG. 10). As manipulation portion 10183b is pivoted towards housing 110 (e.g., towards the first or second actuated positions), first leg 10185a is moved towards second leg 10185b to increasingly tension biasing spring 10184 and build up potential energy in biasing spring 10184. Thus, upon release of manipulation portion 10183b, activation button 10182 is urged back towards the un-actuated position under the bias of biasing spring 10184.

Continuing with reference to FIG. 10, sensor element 10186 may be a magnetic element, e.g., a metallic (steel, for example) pin, disposed on or within activation button 10182. First and second sensors 10188a, 10188b may be hall sensors or other suitable non-contact switches configured to sense the presence of sensor element 10186. Thus, when activation button 1082 is moved from the un-activated position to the first activated position, first sensor 10188a senses the presence of sensor element 10186, thus enabling power source 130 and/or surgical generator 140 (see FIG. 3) to determine that activation button 10182 is disposed in the first activated position and to activate the corresponding mode of operation. Likewise, when activation button 10182 is moved to the second activated position, second sensor 10188b senses the presence of sensor element 10186, thus enabling power source 130 and/or surgical generator 140 (see FIG. 3) to determine that activation button 10182 is disposed in the second activated position and to activate the corresponding mode of operation.

Tactile assembly 10230 includes a clicker tab 10232 and first and second protrusions 10236a, 10236b. Clicker tab 10232 may be configured as a leaf spring and is attached to activation button 10282 within housing 110 at a first end of clicker tab 10232. Clicker tab 10232 extends from activation button 10282 to a second, free end of clicker tab 10232 within housing 110. First and second protrusions 10236a, 10236b are positioned adjacent to respective first and second sensors 10188a, 10188b along the travel path of actuation button 10182. More specifically, protrusions 10236a, 10236b and sensors 10188a, 10188b are positioned relative to clicker tab 10232 and sensor element 10186, respectively, such that, upon actuation of activation button 10282, the second, free end of clicker tab 10232 contacts, deflects, and ultimately traverses first protrusion 10236a, thereby producing a first audible and/or tactile click, just prior to or contemporaneously with sensor element 10186 moving into position to be detected by first sensor 10188a. Thus, the first audible and/or tactile click produced by clicker tab 10232 indicates to the user that the first activated position is about to be achieved or has been achieved. Similarly, upon further movement of activation button 10182 from the first activated position to the second activated position, the second, free end of clicker tab 10232 contacts, deflects, and ultimately traverses second protrusion 10236b, thereby producing a second audible and/or tactile click, just prior to or contemporaneously with sensor element 10186 moving into position to be detected by second sensor 10188b. Thus, the second audible and/or tactile click produced by clicker tab 10232 indicates to the user that the second activated position is about to be achieved or has been achieved. The first and second audible and/or tactile clicks, in aspects, may be different from one another to enable distinction between the first and second activated positions.

With reference to FIG. 11, another activation button 11282 provided in accordance with the present disclosure and configured for use, for example, with activation assembly 10180 (FIG. 10), or any other suitable activation assembly, is shown. Activation button 11282 is similar to and may include any of the features of activation button 10282 (FIG. 10) except that leg 11185 (functioning as a leg of a torsion spring) and clicker tab 11232 are integrally formed with activation button 11282 (e.g., monolithically formed from a single-shot mold) as living hinges extending from activation button 11282. Similarly as detailed above with respect to activation assembly 10180 (FIG. 10), leg 11185 is configured to abut stop surface 111b disposed within housing 110 (see FIG. 10) to bias activation button 11282 towards the un-actuated position, and the second, free end of clicker tab 11232 is configured to interact with protrusions 10236a, 10236b (see FIG. 10) to indicate to the user that the first activated position or the second activated position is about to be achieved or has been achieved.

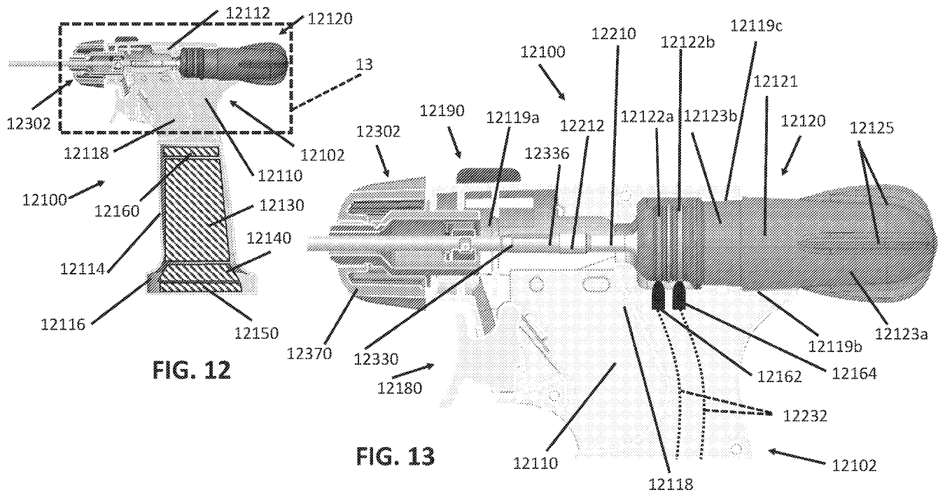

Turning to FIGS. 12 and 13, another cordless ultrasonic surgical instrument provided in accordance with the aspects and features of the present disclosure is shown generally identified by reference numeral 12100. Surgical instrument 12100 is similar to and may include any of the features of surgical instrument 100 (FIGS. 1-3), detailed above. Accordingly, for purposes of brevity, similarities between surgical instrument 12100 and surgical instrument 100 (FIGS. 1-3) are summarily described hereinbelow or omitted entirely. Surgical instrument 12100 differs from surgical instrument 100 (FIGS. 1-3), as detailed below, at least in that ultrasonic transducer assembly 12120 is removable from handle assembly 12102 and separately sterilizable for reuse, rather than being integrated into a sealed compartment defined within handle assembly 12102. Alternatively, ultrasonic transducer assembly 12120 may be integrated with handle assembly 12102 and sterilizable for reuse with handle assembly 12102, e.g., via sealing ultrasonic transducer assembly 12120 itself or sealing ultrasonic transducer assembly 12120 within handle assembly 12102 (such as detailed above, except that ultrasonic transducer assembly 12120 is sealed separately from the sealed compartment(s) retaining the other components within handle assembly 12102).

Surgical instrument 12100 generally includes a handle assembly 12102, an elongate assembly 12302 that is configured to releasably engage handle assembly 12102, and an ultrasonic transducer assembly 12120 that is configured to releasably engage handle assembly 12102 and releasably operably couple to elongate assembly 12302. Handle assembly 12102, in aspects, is configured as a reusable portion. Elongate assembly 12302, in aspects, is configured as a disposable portion. Ultrasonic transducer assembly 12120, in aspects, is configured as a reusable portion such that, after a use of ultrasonic transducer assembly 12120 in a surgical procedure, ultrasonic transducer assembly 12120 is capable of being sterilized (e.g., in an autoclave), cleaned, and/or otherwise prepared for reuse.

Handle assembly 12102 includes a housing 12110 defining a barrel portion 12112 and a fixed handle portion 12114 depending from barrel portion 12112. Housing 12110 includes a sealed compartment 12116 and an exposed shell 12118. Sealed compartment 12116 is defined within fixed handle portion 12114 of handle assembly 12102 and supports and encloses a power source 12130, a surgical generator 12140, a charging interface 12150, and an electrical connection assembly 12160. Sealed compartment 12116 may be hermetically or otherwise sealed to enable handle assembly 12102 to withstand autoclaving and/or other sterilization without damaging the internal components within sealed compartment 12116.

Exposed shell 12118 of handle assembly 12102 operably supports an actuation lever (not shown, see actuation lever 170 (FIGS. 1-3)), an activation assembly 12180, and an engagement mechanism 12190. Exposed shell 12118 is configured to releasably receive a portion of elongate assembly 12302 and engagement mechanism 12190 is configured to releasably engage the portion of elongate assembly 12302 within handle assembly 12102, similarly as detailed herein or in any other suitable manner. Exposed shell 12118 is further configured to releasably receive a portion of ultrasonic transducer assembly 12120 to enable operable engagement of ultrasonic transducer assembly 12120 with elongate assembly 12302 and electrical coupling of ultrasonic transducer assembly 12120 with the electronics (e.g., power source 12130, surgical generator 12140, and/or electrical connection assembly 12160) disposed within sealed compartment 12116 of handle assembly 12102. More specifically, exposed shell 12118 defines a distal nose 12119a through which the portion of elongate assembly 12302 is inserted to releasably engage elongate assembly 12302 with exposed shell 12118, and a proximal cavity 12119b through which the portion of ultrasonic transducer assembly 12120 is inserted to releasably engage ultrasonic transducer assembly 12120 with exposed shell 12118. Distal nose 12119a and proximal cavity 12119b communicate with one another within exposed shell 12118 to enable operable engagement of ultrasonic transducer assembly 12120 and elongate assembly 12302 with one another within exposed shell 12118, as detailed below.

Continuing with reference to FIGS. 12 and 13, ultrasonic transducer assembly 12120 includes a sealed outer housing 12121, an ultrasonic transducer (not shown, see ultrasonic transducer 120 (FIG. 3)) sealed within sealed outer housing 12121, first and second ring contacts 12122a, 12122b disposed annularly about sealed outer housing 12121, longitudinally-spaced relative to one another along sealed outer housing 12121, and electrically connected with electrodes (not shown, see electrodes 126, 128 of ultrasonic transducer 120 (FIG. 3)) of the ultrasonic transducer through sealed outer housing 12121, and an ultrasonic horn 12210 engaged with the ultrasonic transducer within sealed outer housing 12121 and extending from sealed outer housing 12121 to a distal connector 12212, e.g., a threaded female receiver. Sealed outer housing 12121 may further include a proximal knob 12123a defining one or more flutes 12125 or other suitable features to facilitate rotation of proximal knob 12123a and, thus, ultrasonic transducer assembly 12120.

Sealed outer housing 12121 may additionally or alternatively include an annular recess 12123b configured for receipt within an annular protrusion 12119c extending into proximal cavity 12119b of exposed shell 12118 to rotatably engage ultrasonic transducer assembly 12120 within exposed shell 12118, e.g., via snap-fit engagement of annular protrusion 12119c within annular recess 12123b, upon sufficient insertion of ultrasonic transducer assembly 12120 distally into proximal cavity 12119b. However, other suitable releasable rotatable engagements are also contemplated.

With ultrasonic transducer assembly 12120 and elongate assembly 12302 engaged within exposed shell 12118, ultrasonic transducer assembly 12120 and elongate assembly 12302 may be operably engaged with one another by rotating ultrasonic transducer assembly 12120 and elongate assembly 12302 in opposite directions or by maintaining one of ultrasonic transducer assembly 12120 and elongate assembly 12302 in fixed position while rotating the other of ultrasonic transducer assembly 12120 and elongate assembly 12302. In this manner, proximal connector 12336 (e.g., a threaded male shaft) of waveguide 12330 of elongate assembly 12320 is threadingly engaged with distal connector 12212 of ultrasonic horn 12210 of ultrasonic transducer assembly 12120 within exposed shell 12118 such that mechanical ultrasonic motion output from the ultrasonic transducer is transmitted along ultrasonic horn 12210 and waveguide 12330 to the end effector (not shown, see end effector 380 (FIGS. 1 and 2)) for treating tissue therewith.

At least a portion of proximal knob 12123a remains external of exposed shell 12118 when ultrasonic transducer assembly 12120 is engaged within exposed shell 12118 and rotation knob 12370 of elongate assembly 12302 remains external of exposed shell 12118 when elongate assembly 12302 is engaged within exposed shell 12118 such that rotation knob 12370 and/or proximal knob 12123a can be grasped and rotated relative to one another to facilitate the operable engagement of ultrasonic transducer assembly 12120 and elongate assembly 12302 with one another. Further, once ultrasonic transducer assembly 12120 and elongate assembly 12302 are operably engaged, either or both of rotation knob 12370 and proximal knob 12123a may be utilized to rotate ultrasonic transducer assembly 12120 and elongate assembly 12302 together, e.g., to orient the end effector as desired during use.

Referring still to FIGS. 12 and 13, exposed shell 12118 includes first and second contacts 12162, 12164 mounted therein and extending into proximal cavity 12119b of exposed shell 12118. First and second contacts 12162, 12164 may be leaf springs, coil springs, other spring contacts, pogo pins, roller contacts, slip ring contacts, or any other suitable contacts configured to contact first and second ring contacts 12122a, 12122b of ultrasonic transducer assembly 12120 to electrically couple ultrasonic transducer assembly 12120 with the electronics (e.g., power source 12130, surgical generator 12140, and/or electrical connection assembly 12160) of handle assembly 12102 upon engagement of ultrasonic transducer assembly 12120 within exposed shell 12118. Contacts 12162, 12164, more specifically, are configured to maintain electrical connection with first and second ring contacts 12122a, 12122b regardless of the rotational orientation of ultrasonic transducer assembly 12120 relative to exposed shell 12118.

One or more electrical connectors 12232, e.g., electrical wires, electrical contacts, electrically conductive elements, combinations thereof, and/or or other suitable electrical pathways, may be embedded within the material forming housing 12110 and/or disposed within internal sealed conduits within housing 12110 to provide electrical connection to and from sealed compartment 12116 such as, for example, between contacts 12162, 12164 (and, thus, ultrasonic transducer assembly 12120) and electrical connection assembly 12160 (or directly between contacts 12162, 12164 and power source 12130 and/or surgical generator 12140).

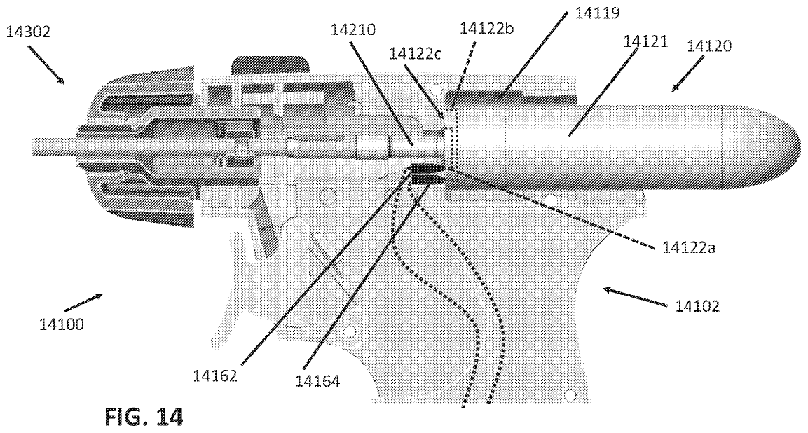

With reference to FIG. 14, another a cordless ultrasonic surgical instrument provided in accordance with the aspects and features of the present disclosure is shown generally identified by reference numeral 14100. Surgical instrument 14100 is similar to and may include any of the features of surgical instrument 12100 (FIGS. 12 and 13), detailed above. Accordingly, for purposes of brevity, only the differences between surgical instrument 14100 and surgical instrument 12100 (FIGS. 12 and 13) are described in detail hereinbelow.

Surgical instrument 14100 generally includes a handle assembly 14102, an elongate assembly 14302 that is configured to releasably engage handle assembly 14102, and an ultrasonic transducer assembly 14120 that is configured to releasably engage handle assembly 14102 and releasably operably couple to elongate assembly 14302.

Ultrasonic transducer assembly 14120 includes a sealed outer housing 14121, an ultrasonic transducer (not shown, see ultrasonic transducer 120 (FIG. 3)) sealed within sealed outer housing 14121, and an ultrasonic horn 14210 engaged with the ultrasonic transducer within sealed outer housing 14121 and extending distally from sealed outer housing 14121. Ultrasonic transducer assembly 14120 further includes first (inner) and second (outer) ring contacts 14122a, 14122b concentrically arranged about ultrasonic horn 14210 (and relative to one another) and disposed on a distal face 14122c of sealed outer housing 14121. Ring contacts 14122a, 14122b are electrically connected with electrodes (not shown, see electrodes 126, 128 of ultrasonic transducer 120 (FIG. 3)) of the ultrasonic transducer through sealed outer housing 14121.

First and second contacts 14162, 14164 are mounted within handle assembly 14102 and extend into proximal cavity 14119 such that first contact 14162 is disposed proximally of and in electrical contact with first ring contact 14122a and such that second contact 14164 is disposed proximally of and in electrical contact with second ring contact 14122b. First and second contacts 14162, 14164 may be leaf springs, coil springs, other spring contacts, pogo pins, roller contacts, slip ring contacts, or any other suitable contacts configured to contact first and second ring contacts 14122a, 14122b of ultrasonic transducer assembly 14120 to electrically couple ultrasonic transducer assembly 14120 with the electronics of handle assembly 14102 upon engagement of ultrasonic transducer assembly 14120 within handle assembly 14102. Contacts 14162, 14164, more specifically, are configured to maintain electrical connection with first and second ring contacts 14122a, 14122b regardless of the rotational orientation of ultrasonic transducer assembly 14120 relative to handle assembly 14102.

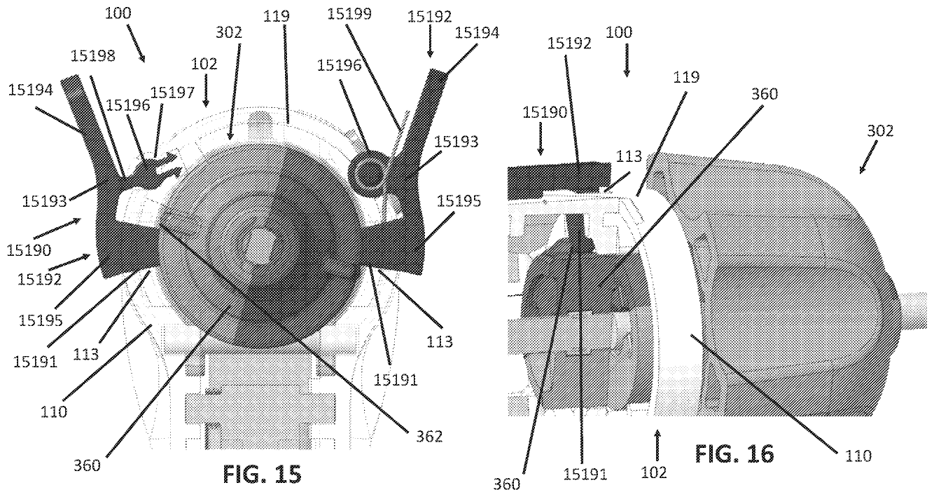

Turning to FIGS. 15 and 16, an engagement mechanism 15190 provided in accordance with the present disclosure is shown disposed at distal nose 119 of housing 110 and configured to enable releasable engagement of elongate assembly 302 with housing 110 of handle assembly 102. Although detailed herein with respect to surgical instrument 100, it is contemplated that engagement mechanism 15190 may be used in conjunction with any of the other surgical instruments detailed herein or any other suitable surgical instrument.

Engagement mechanism 15190 includes opposing lock arms 15192 disposed on opposite sides of distal nose 119 of housing 110, e.g., laterally opposing one another, although vertical opposition is also contemplated. Each lock arm 15192 includes a pivot portion 15193, a manipulation portion 15194 extending from the pivot portion 15193 in a first direction, and an engagement portion 15195 extending from the pivot portion 15193 in a second opposite direction such that pivot portions 15193 of lock arms 15192 are disposed between manipulation portions 15194 and engagement portions 15195.

A pivot boss 15196 is coupled to each pivot portion 15193 and is pivotably captured within a corresponding recess 15197 defined within housing 110 on either side of distal nose 119 of housing 110. Pivot bosses 15196, more specifically, may be connected to respective pivot portions 15193 via living hinges 15198. Thus, with pivot bosses 15196 pivotably coupling pivot potions 15193 of lock arms 15192 to housing 110, and with manipulation portions 15194 and engagement portions 15195 of respective lock arms 15192 disposed on opposing sides of pivot portions 15193, movement of manipulation portions 15194 inwardly towards housing 110 pivots pivot portions 15193 relative to housing 110 to thereby move engagement portions 15195 outwardly away from housing 110, e.g., in an opposite direction relative to the movement of manipulation portions 15194. Similarly, movement of manipulation portions 15194 outwardly away from housing 110 pivots pivot portions 15193 relative to housing 110 and thereby moves engagement portions 15195 inwardly towards housing 110. Further, as spring, e.g., a torsion spring 15199, may be provided to bias lock arms 15192 towards engaged positions (only one torsion spring 15199 is shown in FIG. 15), wherein manipulation portions 15194 extend outwardly away from housing 110 and engagement portion 15195 extend inwardly towards housing 110. Torsion springs 15199 may include, for example, body portions disposed about pivot bosses 15196, first legs disposed in abutting contact with manipulation portions 15194, and second legs disposed in abutting contact with housing 110 in order to provide the above-detailed bias, although other suitable configurations of torsion springs or other suitable springs are also contemplated. As an alternative to pivotable coupling of lock arms 15192 to housing 110 to enable pivoting of lock arms 15192 relative to housing 110, lock arms 15192 may be coupled to housing 110 in any other suitable manner (e.g., via pin-slot engagement) to enable other suitable movement (e.g., sliding movement) of lock arms 15192 relative to housing 110 between the engaged and disengaged positions.

Continuing with reference to FIGS. 15 and 16, manipulation portions 15194 of lock arms 15192 are configured to be manually manipulated by a user, e.g., squeezed inwardly towards housing 110 with a user's fingers. Engagement portions 15195 of lock arms 15192 include engagement tabs 15191 extending therefrom inwardly towards housing 110 in alignment with corresponding slots 113 defined through housing 110 and in communication with the interior of distal nose 119 of housing 110. In the engaged positions of lock arms 15192, engagement tabs 15191 extend through slots 113 and protrude into the interior of distal nose 119 of housing 110. In disengaged positions, on the other hand, wherein manipulation portions 15194 are disposed inwardly towards housing 110 and engagement portions 15195 are disposed outwardly away from housing 110, engagement tabs 15191 are withdrawn at least partially into slots 113 such that engagement tabs 15191 do not protrude into or protrude minimally into (e.g., so as not to interfere with elongate assembly 302) the interior of distal nose 119 of housing 110.

Elongate assembly 302, as noted above, includes a nozzle 360 configured for insertion into distal nose 119 of housing 110 and defining an annular channel 362. More specifically, the annular channel 362 of elongated assembly 302 may be a continuous annular channel extending about at least a portion of a circumference thereof or plural discrete annular channels disposed about a circumference of nozzle 360. In either configuration, with nozzle 360 of elongate assembly 302 inserted into distal nose 119 of housing 110, slots 113 defined within housing 110 are aligned with the annular channel 362 such that, upon movement of lock arms 15192 to the engaged positions, engagement tabs 15191 extend through slots 113, into the interior of distal nose 119 of housing 110, and into engagement within annular channel 362 to thereby rotatably engage elongate assembly 302 with housing 110 of handle assembly 102. As noted above, lock arms 15192 may be biased towards these engaged positions. As such, a user may move lock arms 15192 to the disengaged positions against the biases thereof prior to insertion or removal of elongate assembly 302 into or from housing 110. Alternatively, as detailed above, a cam surface or surfaces associated with elongate assembly 302 may urge lock arms 15192 from the engaged positions towards the disengaged positions upon insertion of elongate assembly 302 into housing 110 to permit the insertion of elongate assembly 302 into housing 110. With lock arms 15192 disposed in the disengaged positions (whether by the user moving lock arms 15192 or elongate assembly 302 urging lock arms 15192) and elongate assembly 302 inserted into distal nose 119 of housing 110, lock arms 15192 may return under bias and/or may be released, allowing lock arms 15192 to return to the engaged positions under the biases thereof, thereby engaging elongate assembly 302 with housing 110 of handle assembly 102.

The rotatable engagement of elongate assembly 302 with housing 110 enables rotation of elongate assembly 302 relative to housing 110 without compromising the engagement therebetween. Further, in aspects, the above-detailed engagement of elongate assembly 302 with housing 110 enables insertion of elongate assembly 302 into distal nose 119 and engagement with of elongate assembly 302 housing 110 in any rotational orientation of elongated assembly 302. Alternatively, clocking features may be provided on elongate assembly 302 and/or housing 110 to require insertion of elongate assembly 302 in a defined orientation (or in one of a number of defined orientations, e.g., first and second orientations 180 degrees apart) in order to enable engagement of elongate assembly 302 with housing 110.

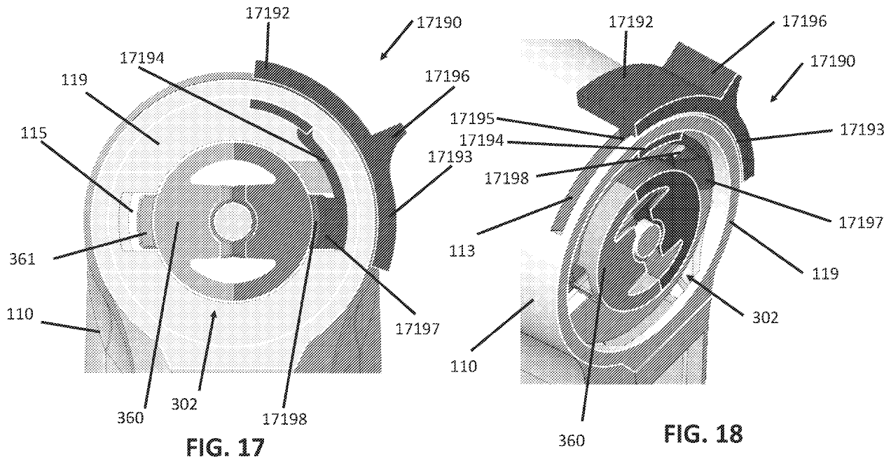

Referring to FIGS. 17 and 18, another engagement mechanism 17190 provided in accordance with the present disclosure is shown disposed at distal nose 119 of housing 110 and configured to enable releasable engagement of nozzle 360 of elongate assembly 302 (FIGS. 1-3 and 6) with housing 110. Although detailed herein with respect to surgical instrument 100 (FIGS. 1-3), it is contemplated that engagement mechanism 17190 may be used in conjunction with any of the other surgical instruments detailed herein or any other suitable surgical instrument.

Engagement mechanism 17190 includes a slider 17192 captured within a slot 113 defined through distal nose 119 of housing 110 and extending annularly about a portion of a circumference of distal nose 119. Slider 17192, more specifically, includes a manipulation portion 17193 disposed on an external surface of housing 110, an engagement portion 17194 disposed within housing 110, and a connector 17195 extending through slot 113 to interconnect manipulation portion 17193 and engagement portion 17194 with one another. Manipulation portion 17193 may include a finger tab 17196 and/or other suitable feature configured to facilitate manual sliding of slider 17192 annularly about a portion of a circumference of distal nose 119 from a disengaged position to an engaged position. Engagement portion 17194 includes at least one first engagement tab 17197 extending inwardly into distal nose 119 of housing 110.

Elongate assembly 302, as noted above, includes a nozzle 360 configured for insertion into distal nose 119 of housing 110. Nozzle 360 defines at least one second engagement tab 17198 extending radially outwardly from nozzle 360. With nozzle 360 of elongate assembly 302 inserted into distal nose 119 of housing 110, the at least one second engagement tab 17198 is positioned adjacent the circumferential travel path of the at least one first engagement tab 17197 such that, upon sliding of slider 17192 from the disengaged position to the engaged position, the at least one first engagement tab 17197 is moved into engagement with the at least one second engagement tab 17198 to thereby engage elongate assembly 302 with housing 110.

With slider 17192 disposed in the disengaged position, the at least one first engagement tab 17197 is displaced from the at least one second engagement tab 17198 or the travel path thereof such that elongate assembly 302 can be inserted into or withdrawn from distal nose 119 of housing 110. However, as noted above, with nozzle 360 of elongate assembly 302 inserted into distal nose 119 of housing 110 and slider 17192 disposed in the engaged position, the engagement tabs 17197, 17198 are engaged with one another, thereby engaging elongate assembly 302 with housing 110. In aspects, a biasing spring (not shown) is provided to bias slider 17192 towards the engaged position such that the user is required to move slider 17192 against the bias towards the disengaged position to enable insertion and removal of elongate assembly 302 and can release slider 17192 with elongated assembly 302 positioned within distal nose 119 to enable engagement of elongate assembly 302 with housing 110. As an alternative to a user moving slider 17192 to the disengaged position to permit insertion of elongate assembly 302, elongate assembly 302 may be configured to contact and cam slider 17192 from the engaged position to the disengaged position, similarly as detailed above, thus obviating the need for the user to move slider 17192 in order to insert elongate assembly 302.

In aspects, engagement mechanism 17190 rotatably engages at least a portion of elongate assembly 302 with housing 110 to enable rotation of at least a portion of elongate assembly 302 relative to housing 110 without compromising the engagement therebetween. In such aspects, for example, nozzle 360 may engage housing 110 in fixed rotational orientation while the remainder of elongate assembly 302 is rotatably coupled to nozzle 360 to thereby permit rotation of the remainder of elongate assembly 302 relative to housing 110 when nozzle 360 is engaged with housing 110. Further, clocking tabs 361 and corresponding clocking slots 115 or other suitable features may be provided on nozzle 360 of elongate assembly 302 and within housing 110, respectively, to ensure insertion of elongate assembly 302 in a defined orientation (or in one of a number of defined orientations, e.g., first and second orientations 180 degrees apart) relative to housing 110 in order to enable engagement of elongate assembly 302 with housing 110. In other aspects, nozzle 360 may be inserted into and engaged within housing 110 in any orientation.

As an alternative to the above-detailed configuration wherein slider 17192 is configured to slide annularly about a portion of the circumference of distal nose 119 between the engaged and disengaged positions, slider 17192 may be configured to slide longitudinally (e.g., in proximal and distal directions) along a portion of distal nose 119 between the engaged and disengaged positions. In such configurations, locking and unlocking is achieved similarly as detailed above except that tabs 17197, 17198 are re-oriented to enable locking an unlocking in response to longitudinal sliding of slider 17192.

Aspects of this disclosure may be further described by reference to the following numbered paragraphs:
1. A surgical instrument, comprising: a housing including a sealed compartment and an exposed shell, the housing configured to withstand autoclave sterilization and configured to releasably engage an elongate assembly including an end effector; electronics sealed within the sealed compartment of the housing, wherein the sealed compartment protects the electronics to enable autoclave sterilization of the housing without damaging the electronics; and an activation assembly operably coupled to the exposed shell of the housing, the activation assembly including an activation button movable relative to the housing between an un-activated position and at least one activated position, and one or more electrical switches configured to detect activation of the activation button and, the at least one electrical switch electrically coupled to the electronics and configured to transmit the detected activation to the electronics, wherein, with the elongate assembly engaged with the housing, the electronics, in response to receipt of the detected activation, are configured to energize the end effector of the elongate assembly to treat tissue with energy.
2. The surgical instrument according to paragraph 1, wherein the activation assembly further includes a tactile assembly configured to provide a perceptible output indicating a position of the activation button.
3. The surgical instrument according to paragraph 2, wherein the tactile assembly includes: at least one clicker tab coupled to the housing and extending into an actuation path of the activation button such that the at least one clicker tab provides the perceptible output in response to movement of the activation button from the un-activated position to one of the at least one activated positions; at least one bistable spring disposed in the actuation path of the activation button such that the at least one bistable spring provides the perceptible output in response to movement of the activation button from the un-activated position to one of the at least one activated positions; and/or at least one mechanical dome disposed in the actuation path of the activation button such that the at least one mechanical dome provides the perceptible output in response to movement of the activation button from the un-activated position to one of the at least one activated positions.
4. The surgical instrument according to any preceding paragraph, wherein the activation button further includes a biasing spring integrally formed with the activation button.
5. The surgical instrument according to any preceding paragraph, further comprising: an engagement mechanism operably coupled to the exposed shell of the housing, the engagement mechanism configured to releasably engage the elongate assembly with the housing, the engagement mechanism configured to withstand autoclave sterilization.
6. The surgical instrument according to paragraph 5, further comprising at least one contact disposed within the exposed shell and electrically coupled to the electronics, the at least one contact configured to detect engagement of the elongate assembly with the housing.
7. The surgical instrument according to paragraph 6, wherein the electronics are configured to at least one of read information from or write information to the elongate assembly via the at least one contact.
8. The surgical instrument according to any one of paragraphs 5-7, wherein the engagement mechanism includes one of: at least one lock arm pivotable relative to the housing between a disengaged position, permitting insertion and removal of the elongate assembly to and from the housing, and an engaged position, engaging the elongate assembly with the housing; or a slider slidable about a circumference of the housing between a disengaged position, permitting insertion and removal of the elongate assembly to and from the housing, and an engaged position, engaging the elongate assembly with the housing.
9. The surgical instrument according to any preceding paragraph, wherein the electronics include an ultrasonic generator and an ultrasonic transducer.
10. The surgical instrument according to paragraph 9, further comprising an ultrasonic horn engaged with the ultrasonic transducer within the sealed compartment and extending into the exposed shell, the ultrasonic horn defining a distal connector disposed within the exposed shell and configured to releasably engage an ultrasonic waveguide of the elongate assembly.
11. The surgical instrument according to any one of paragraphs 1-8, wherein the electronics include an ultrasonic generator, and wherein the surgical instrument further includes an ultrasonic transducer assembly releasably rotatably engagable with the housing.
12. The surgical instrument according to paragraph 11, further comprising first and second electrical contacts disposed within the exposed shell of the housing and electrically coupled to the ultrasonic generator, wherein, with the ultrasonic transducer assembly rotatably engaged with the housing, the first and second electrical contacts are configured to maintain electrical contact with first and second ring contacts, respectively, of the ultrasonic transducer assembly regardless of a rotational orientation of the ultrasonic transducer assembly relative to the housing.
13. The surgical instrument according to paragraph 12, wherein the first and second ring contacts are disposed annularly about a circumference of a portion of the ultrasonic transducer assembly or disposed concentrically about a distal face of a portion of the ultrasonic transducer assembly.
14. The surgical instrument according to any preceding paragraph, further comprising an actuation lever operably coupled to the exposed shell of the housing, the actuation lever configured to couple to the elongate assembly when the elongate assembly is engaged with the housing and configured to mechanically actuate the end effector of the elongate assembly when the elongate assembly is engaged with the housing, wherein the actuation lever is configured to withstand autoclave sterilization.
15. The surgical instrument according to any preceding paragraph, wherein the electronics include a power source and a wireless charging interface for charging the power source.
16. A surgical instrument, comprising: a housing including a sealed compartment and an exposed shell, the housing configured to withstand autoclave sterilization and configured to releasably engage an elongate assembly including an end effector; electronics sealed within the sealed compartment of the housing, wherein the sealed compartment protects the electronics to enable autoclave sterilization of the housing without damaging the electronics; and an activation assembly operably coupled to the exposed shell of the housing, the activation assembly including an activation button movable relative to the housing between an un-activated position and at least one activated position, a tactile assembly configured to provide a perceptible output indicating a position of the activation button, and at least one electrical switch configured to detect activation of the activation button, the at least one electrical switch electrically coupled to the electronics and configured to transmit the detected activation to the electronics, wherein, with the elongate assembly engaged with the housing, the electronics, in response to receipt of the detected activation, are configured to energize the end effector of the elongate assembly to treat tissue with energy, and wherein the activation assembly is configured to withstand autoclave sterilization.
17. The surgical instrument according to paragraph 16, wherein the tactile assembly includes at least one clicker tab coupled to the housing and extending into an actuation path of the activation button such that the at least one clicker tab provides the perceptible output in response to movement of the activation button from the un-activated position to one of the at least one activated positions.
18. The surgical instrument according to paragraph 16, wherein the tactile assembly includes at least one bistable spring disposed in an actuation path of the activation button such that the at least one bistable spring provides the perceptible output in response to movement of the activation button from the un-activated position to one of the at least one activated positions.
19. The surgical instrument according to paragraph 16, wherein the tactile assembly includes at least one mechanical dome disposed in an actuation path of the activation button such that the at least one mechanical dome provides the perceptible output in response to movement of the activation button from the un-activated position to one of the at least one activated positions.
20. The surgical instrument according to paragraph 16, wherein the tactile assembly includes a clicker tab coupled to the activation button and configured to interact with a portion of the housing in response to movement of the activation button from the un-activated position to one of the at least one activated positions such that the at least one clicker tab provides the perceptible output in response to the movement of the activation button from the un-activated position to the one of the at least one activated positions.
21. The surgical instrument according to paragraph 20, wherein the clicker tab is integrally formed with the activation button.
22. The surgical instrument according to any preceding paragraph, wherein the activation button further includes a biasing spring integrally formed with the activation button.
23. A surgical instrument, comprising: a housing including a sealed compartment and an exposed shell, the housing configured to withstand autoclave sterilization; electronics sealed within the sealed compartment of the housing, wherein the sealed compartment protects the electronics to enable autoclave sterilization of the housing without damaging the electronics; an engagement mechanism operably coupled to the exposed shell of the housing, the engagement mechanism configured to releasably engage an elongate assembly with the housing, the engagement mechanism configured to withstand autoclave sterilization; and an actuation lever operably coupled to the exposed shell of the housing, the actuation lever configured to couple to the elongate assembly when the elongate assembly is engaged with the housing and configured to mechanically actuate an end effector of the elongate assembly when the elongate assembly is engaged with the housing, wherein the actuation lever is configured to withstand autoclave sterilization, wherein, when the elongate assembly is engaged with the housing, the electronics are configured to energize the end effector of the elongate assembly to treat tissue with energy.
24. The surgical instrument according to paragraph 23, further comprising at least one contact disposed within the exposed shell and electrically coupled to the electronics, the at least one contact configured to detect engagement of the elongate assembly with the housing.
25. The surgical instrument according to paragraph 24, wherein the electronics are configured to at least one of read information from or write information to the elongate assembly via the at least one contact.
26. The surgical instrument according to paragraph 23, wherein the electronics include an ultrasonic generator and an ultrasonic transducer.
27. The surgical instrument according to paragraph 26, further comprising an ultrasonic horn engaged with the ultrasonic transducer within the sealed compartment and extending into the exposed shell.
28. The surgical instrument according to claim 27, wherein the ultrasonic horn defines a distal connector disposed within the exposed shell and configured to releasably engage an ultrasonic waveguide of the elongate assembly.
29. The surgical instrument according to paragraph 23, wherein the electronics include an ultrasonic generator, and wherein the surgical instrument further includes an ultrasonic transducer assembly releasably rotatably engagable with the housing.
30. The surgical instrument according to paragraph 29, further comprising first and second electrical contacts disposed within the exposed shell of the housing and electrically coupled to the ultrasonic generator, wherein, with the ultrasonic transducer assembly rotatably engaged with the housing, the first and second electrical contacts are configured to maintain electrical contact with first and second ring contacts, respectively, of the ultrasonic transducer assembly regardless of a rotational orientation of the ultrasonic transducer assembly relative to the housing.
31. The surgical instrument according to paragraph 30, wherein the first and second ring contacts are disposed annularly about a circumference of the ultrasonic transducer assembly.
32. The surgical instrument according to paragraph 30, wherein the first and second ring contacts are concentrically disposed about an ultrasonic horn of the ultrasonic transducer assembly on a distal face of the ultrasonic transducer assembly.
33. The surgical instrument according to any one of paragraphs 23-32, wherein the engagement mechanism includes at least one lock arm movable relative to the housing between a disengaged position, permitting insertion and removal of the elongate assembly to and from the housing, and an engaged position, engaging the elongate assembly with the housing.
34. The surgical instrument according to paragraph 33, wherein the engagement mechanism further includes at least one biasing spring biasing the at least one lock arm towards the engaged position.
35. The surgical instrument according to any one of paragraphs 23-32, wherein the engagement mechanism includes a slider slidable about a circumference of the housing between a disengaged position, permitting insertion and removal of the elongate assembly to and from the housing, and an engaged position, engaging the elongate assembly with the housing.
36. A surgical instrument, comprising: a reusable portion, including: a housing including a sealed compartment and an exposed shell, the housing configured to withstand autoclave sterilization; electronics sealed within the sealed compartment of the housing, wherein the sealed compartment protects the electronics to enable autoclave sterilization of the housing without damaging the electronics; an engagement mechanism operably coupled to the exposed shell of the housing, the engagement mechanism configured to releasably engage an elongated assembly with the housing, the engagement mechanism configured to withstand autoclave sterilization; an actuation lever operably coupled to the exposed shell of the housing, the actuation lever configured to couple to the elongated assembly when the elongated assembly is engaged with the housing, the actuation lever configured to mechanically actuate an end effector of the elongated assembly, wherein the actuation lever is configured to withstand autoclave sterilization; and an activation assembly operably coupled to the exposed shell of the housing, the activation assembly including an activation button movable relative to the housing between an un-activated position and at least one activated position, and one or more electrical switches configured to detect activation of the activation button and, the at least one electrical switch electrically coupled to the electronics and configured to transmit the detected activation to the electronics, wherein the activation assembly is configured to withstand autoclave sterilization.
37. The surgical instrument according to paragraph 36, wherein the electronics include a surgical generator and an ultrasonic transducer.
38. The surgical instrument according to paragraph 37, wherein the reusable portion further includes an ultrasonic horn engaged with the ultrasonic transducer within the sealed compartment and extending into the exposed shell.
39. The surgical instrument according to paragraph 38, wherein the ultrasonic horn defines a distal connector disposed within the exposed shell and configured to releasably engage an ultrasonic waveguide of the elongated assembly.
40. The surgical instrument according to paragraph 38 or 39, wherein the reusable portion further includes a divider disposed within the housing and separating the sealed compartment and the exposed shell from one another, wherein the ultrasonic horn extends through the divider.
41. The surgical instrument according to any one of paragraphs 37-40, wherein the ultrasonic transducer is rotatable relative to the housing.
42. The surgical instrument according to any one of paragraphs 36-41, wherein the reusable portion further includes at least one electrical connector electrically coupled to the electronics within the sealed compartment and extending into the exposed shell.
43. The surgical instrument according to paragraph 42, wherein the electronics include a surgical generator and the at least one electrical connector includes first and second electrical connectors configured to deliver bipolar energy to the end effector of the elongated assembly.
44. The surgical instrument according to any one of paragraphs 36-43, wherein the activation assembly further includes a tactile assembly configured to provide a perceptible output indicating a position of the activation button.
45. The surgical instrument according to paragraph 44, wherein the reusable portion further includes at least one contact disposed within the exposed shell and electrically coupled to the electronics, the at least one contact configured to detect engagement of the elongated assembly with the housing.
46. The surgical instrument according to paragraph 45, wherein the electronics are configured to at least one of read information from or write information to the elongated assembly via the at least one contact.
47. The surgical instrument according to any one of paragraphs 36-46, wherein each of the one or more electrical switches is a non-contact switch.
48. The surgical instrument according to any one of paragraphs 36-47, further comprising a disposable portion configured to releasably engage the reusable portion, the disposable portion including the elongated assembly.
49. The surgical instrument according to any one of paragraphs 36-48, wherein the electronics include a power source and a wireless charging interface for charging the power source.
50. A surgical instrument, comprising: a reusable portion, including: a housing configured to withstand autoclave sterilization; electronics disposed within the housing; an engagement mechanism operably coupled to the housing, the engagement mechanism configured to releasably engage an elongated assembly with the housing, the engagement mechanism configured to withstand autoclave sterilization; and an activation assembly operably coupled to the housing, the activation assembly configured to withstand autoclave sterilization and including: an activation button movable relative to the housing between an un-activated position and at least one activated position; one or more non-contact electrical switches configured to detect activation of the activation button, the one or more non-contact electrical switches electrically coupled to the electronics and configured to transmit the detected activation to the electronics, the electronics configured to initiate a supply of energy to an end effector of the elongated assembly in response to the detected activation; and a tactile assembly configured to provide a perceptible output indicating a position of the activation button.
51. The surgical instrument according to paragraph 50, wherein the tactile assembly includes at least one clicker tab disposed in an actuation path of the activation button such that the at least one clicker tab provides the perceptible output in response to movement of the activation button from the un-activated position to one of the at least one activated positions.
52. The surgical instrument according to paragraph 51, wherein the tactile assembly includes at least one bistable spring disposed in an actuation path of the activation button such that the at least one bistable spring provides the perceptible output in response to movement of the activation button from the un-activated position to one of the at least one activated positions.
53. The surgical instrument according to paragraph 51, wherein the tactile assembly includes at least one mechanical dome disposed in an actuation path of the activation button such that the at least one mechanical dome provides the perceptible output in response to movement of the activation button from the un-activated position to one of the at least one activated positions.
54. The surgical instrument according to any one of paragraphs 50-53, wherein the tactile assembly is exposed within the housing and wherein at least a portion of the one or more non-contact electrical switches is sealed within the housing.
55. The surgical instrument according to any one of paragraphs 50-54, further comprising a disposable portion configured to releasably engage the reusable portion, the disposable portion including the elongated assembly.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

1. A surgical instrument, comprising:
a housing including a sealed compartment and an exposed shell, the housing configured to withstand autoclave sterilization and configured to releasably engage an elongate assembly including an end effector;
electronics sealed within the sealed compartment of the housing, wherein the sealed compartment protects the electronics to enable autoclave sterilization of the housing without damaging the electronics; and
an activation assembly operably coupled to the exposed shell of the housing, the activation assembly including an activation button movable relative to the housing between an un-activated position and at least one activated position, and one or more electrical switches configured to detect activation of the activation button and, the at least one electrical switch electrically coupled to the electronics and configured to transmit the detected activation to the electronics,
wherein, with the elongate assembly engaged with the housing, the electronics, in response to receipt of the detected activation, are configured to energize the end effector of the elongate assembly to treat tissue with energy.

2. The surgical instrument according to claim 1, wherein the activation assembly further includes a tactile assembly configured to provide a perceptible output indicating a position of the activation button.

3. The surgical instrument according to claim 2, wherein the tactile assembly includes:
at least one clicker tab coupled to the housing and extending into an actuation path of the activation button such that the at least one clicker tab provides the perceptible output in response to movement of the activation button from the un-activated position to one of the at least one activated positions;
at least one bistable spring disposed in the actuation path of the activation button such that the at least one bistable spring provides the perceptible output in response to movement of the activation button from the un-activated position to one of the at least one activated positions; and/or
at least one mechanical dome disposed in the actuation path of the activation button such that the at least one mechanical dome provides the perceptible output in response to movement of the activation button from the un-activated position to one of the at least one activated positions.

4. The surgical instrument according to claim 1, wherein the activation button further includes a biasing spring integrally formed with the activation button.

5. The surgical instrument according to claim 1, further comprising:
an engagement mechanism operably coupled to the exposed shell of the housing, the engagement mechanism configured to releasably engage the elongate assembly with the housing, the engagement mechanism configured to withstand autoclave sterilization.

6. The surgical instrument according to claim 5, further comprising at least one contact disposed within the exposed shell and electrically coupled to the electronics, the at least one contact configured to detect engagement of the elongate assembly with the housing.

7. The surgical instrument according to claim 6, wherein the electronics are configured to at least one of read information from or write information to the elongate assembly via the at least one contact.

8. The surgical instrument according to claim 5, wherein the engagement mechanism includes one of:
at least one lock arm pivotable relative to the housing between a disengaged position, permitting insertion and removal of the elongate assembly to and from the housing, and an engaged position, engaging the elongate assembly with the housing; or
a slider slidable about a circumference of the housing between a disengaged position, permitting insertion and removal of the elongate assembly to and from the housing, and an engaged position, engaging the elongate assembly with the housing.

9. The surgical instrument according to claim 1, wherein the electronics include an ultrasonic generator and an ultrasonic transducer.

10. The surgical instrument according to claim 9, further comprising an ultrasonic horn engaged with the ultrasonic transducer within the sealed compartment and extending into the exposed shell, the ultrasonic horn defining a distal connector disposed within the exposed shell and configured to releasably engage an ultrasonic waveguide of the elongate assembly.

11. The surgical instrument according to claim 1, wherein the electronics include an ultrasonic generator, and wherein the surgical instrument further includes an ultrasonic transducer assembly releasably rotatably engagable with the housing.

12. The surgical instrument according to claim 11, further comprising first and second electrical contacts disposed within the exposed shell of the housing and electrically coupled to the ultrasonic generator, wherein, with the ultrasonic transducer assembly rotatably engaged with the housing, the first and second electrical contacts are configured to maintain electrical contact with first and second ring contacts, respectively, of the ultrasonic transducer assembly regardless of a rotational orientation of the ultrasonic transducer assembly relative to the housing.

13. The surgical instrument according to claim 12, wherein the first and second ring contacts are disposed annularly about a circumference of a portion of the ultrasonic transducer assembly or disposed concentrically about a distal face of a portion of the ultrasonic transducer assembly.

14. The surgical instrument according to claim 1, further comprising an actuation lever operably coupled to the exposed shell of the housing, the actuation lever configured to couple to the elongate assembly when the elongate assembly is engaged with the housing and configured to mechanically actuate the end effector of the elongate assembly when the elongate assembly is engaged with the housing, wherein the actuation lever is configured to withstand autoclave sterilization.

15. The surgical instrument according to claim 1, wherein the electronics include a power source and a wireless charging interface for charging the power source.
